(19) United States
(12) Patent Application Publication
(10) Pub. No.: US 2026/0194522 A1
(43) Pub. Date: Jul. 9, 2026

(54) METHODS, SYSTEMS, AND ARRAYS FOR TICKBORNE PATHOGEN AND DISEASE ANALYSIS

(71) Applicant: Vibrant Holdings, LLC, Santa Clara, CA (US)

(72) Inventors: Karthik KRISHNA, San Ramon, CA (US); Hari Krishnan KRISHNAMURTHY, San Jose, CA (US); Vasanth JAYARAMAN, Sunnyvale, CA (US); Kang BEI, San Mateo, CA (US); Tianhao WANG, Fremont, CA (US); John J. RAJASEKARAN, Hillsborough, CA (US)

(21) Appl. No.: 19/558,165

(22) Filed: Mar. 5, 2026

Related U.S. Application Data
(63) Continuation of application No. PCT/US2024/045681, filed on Sep. 6, 2024.
(60) Provisional application No. 63/537,449, filed on Sep. 8, 2023.

(51) Int. Cl. G01N 33/569

(52) U.S. Cl. CPC G01N 33/56911; G01N 33/56905; G01N 33/56983; G01N 33/56994; G01N 2333/015; G01N 2333/035; G01N 2333/045; G01N 2333/05; G01N 2333/085; G01N 2333/183; G01N 2333/185; G01N 2333/20; G01N 2333/44; G01N 2333/45

(57) ABSTRACT

Provided here in are microarrays for analysis and diagnosis of samples containing tickborne pathogens. Also provided herein are methods of use of the microarrays for diagnosis of tickborne-pathogen diseases or conditions.

### CROSS REFERENCE TO RELATED APPLICATIONS

This application is a bypass continuation which claims benefit to PCT/US2024/045681, filed on Sep. 6, 2024, which claims the benefit of U.S. Provisional Application No. 63/537,449, filed Sep. 8, 2023, which is hereby incorporated in its entirety by reference.

### BACKGROUND

Tick-borne infections are the most common vector-borne diseases in the USA. Ticks harbor and spread several infections with Lyme disease being the most common tickborne infection in the US and Europe. Ticks have been shown to transmit more than one infectious agent in a single bite. For instance, there is a 30% chance of getting infected with A. phagocytophilum and a 24% chance of getting infected with B. microti along with Lyme disease after a tick bite. Lack of awareness about tick populations, specific diagnostic tests, and overlapping symptoms of tick-borne infections can often lead to misdiagnosis affecting treatment and the prevalence data reported, especially for non-Lyme tick-borne infections.

Currently, multi-tiered testing is carried out for diagnosing tick-borne infections. In this method, the infectious agents are tested sequentially starting with Lyme disease. This method is time-consuming and can often lead to delayed diagnosis. Testing for multiple infections in a single run helps physicians to arrive at an accurate diagnosis especially when Lyme disease shares symptoms with other vector-borne co-infections. The existing diagnostic assays possess various limitations that restrict their applicability in the diagnosis of these infections. The serial diagnosis of various pathogens is time-consuming and expensive. The diagnosis of Lyme disease and other infections using several blot-based and single-plex ELISA tests remain rudimentary in terms of arriving at a diagnostic conclusion. Additionally, blot-based assays may have overlapping proteins with similar mass requiring additional testing to tease out the specific antigen to which the antibody is bound. Thus, the diagnostic tests currently available for tick-borne diseases are severely limited in their ability to provide accurate results and cannot detect multiple pathogens in a single run. Thus, additional analysis and diagnostic tests for tick-borne diseases are needed.

### SUMMARY

In one aspect, provided herein are microarrays comprising a surface and two or more features attached to the surface, wherein the features comprise a probe molecule from one or more tickborne pathogens and wherein the probe molecules comprise two or more of a lysate, an antigen, a protein, or a peptide.

In some embodiments, the antigen comprises an antigenic fragment.

In some embodiments, the two or more probe molecules are distinct from each other.

In some embodiments, the two or more probe molecules comprise at least one lysate, at least one protein, and/or at least one peptide.

In some embodiments, the microarray is configured to have an at least 25%, 55%, 60%, 65%, 70%, 75%, 80%, 85%, 90%, 91%, 92%, 93%, 94%, 95%, 96%, 97%, 98%, 99%, or 100% sensitivity for detection of a tickborne pathogen after contact of the features with a sample from a subject suspected of having a tickborne pathogen.

In some embodiments, the microarray is configured to have an at least 90%, 91%, 92%, 93%, 94%, 95%, 96%, 97%, 98%, 99%, or 100% specificity for detection of a tickborne pathogen after contact of the features with a sample from a subject suspected of having a tickborne pathogen.

In some embodiments, the microarray is configured to have at least 90% sensitivity and 90% specificity for detection of a tickborne pathogen after contact of the features with a sample from a subject suspected of having a tickborne pathogen.

In some embodiments, the microarray is configured to have at least 93% sensitivity and 96% specificity for detection of a tickborne pathogen after contact of the features with a sample from a subject suspected of having a tickborne pathogen.

In some embodiments, the microarray is configured to have at least 95% sensitivity and 95% specificity for detection of a tickborne pathogen after contact of the features with a sample from a subject suspected of having a tickborne pathogen.

In some embodiments, the one or more tickborne pathogens comprises Borrelia burgdorferi, Borrelia mayonii, Borrelia afzelii, Borrelia garinii, Borrelia Bavariensis, Borrelia speielmanii, Borrelia hemsii, Borrelia Turicatae, Borrelia miyamotoi, Borrelia andersonii, Borrelia maritima, Borrelia californiensis, Borrelia bissettiae, Borrelia lusitaniae, Borrelia valaisiana, Borrelia yangtzensis, Borrelia turcia, Borrelia lonestari, Babsia microti, Babsia duncani, Bartonella henselae, Anaplasma phagocytophilum, Ehrlichia chaffeensis, Rickettsia typhi, Rickettsia rickettsia, Rickettsia parkeri, Francisella tularensis, Powassan virus, Tick-borne encephalitis virus, West Nile virus, Coxsackie virus, Cytomegalovirus, Epstein Barr virus, Parvovirus B19, Toxoplasma, gondii, Colorado tick fever virus, Heartland virus, Bourbon virus, HSV-1, HSV-2, HHV-6, or HHV-7.

In some embodiments, the one or more tickborne pathogens is selected from Tables 1, 3, or A.

In some embodiments, the one or more tickborne pathogen comprises Borrelia burgdorferi.

In some embodiments, the microarray comprises two or more probe molecules from distinct tickborne pathogens.

In some embodiments, the microarray comprises two or more probe molecules from at least 1, 2, 3, 4, 5, 6, 7, 8, 9, 10, 11, 12, 13, 14, 15, 16, 17, 18 or more tickborne pathogens.

In some embodiments, the microarray comprises two or more probe molecules from the same tickborne pathogen.

In some embodiments, the array comprises at least 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 32, 330, 340, 350, 360, 370, 380, 390, 400 or more features comprising probe molecules from one or more tickborne pathogen.

In some embodiments, the microarray comprises at least four hundred or more probe molecules from one or more tickborne pathogen.

In some embodiments, the two or more probe molecules is selected from Table 3 or Table B.

In some embodiments, the two or more probe molecules comprise:
i. B. burgdorferi VLsE1, C6 peptide, DbpB, OspC, p18, p32, p28, p30, p31, OspA, OspB, BmpA, p34, p39, P41, p45, p58, p66, p83, p84, p85 p86, p87, p88, p89, p90, p91, p92, p93, whole cell sonicate crude extract B31, or whole cell sonicate crude extract B297;
ii. B. afzelii BmpA, DbpA, OspA, OscpC, or p100;
iii. B. garinii DBpA or OspC;
iv. B. bavariensis p58, VLsE1, DbpA;
v. B. spielmanii DBpA or OspC;
vi. B. miyamotoi GlpQ;
vii. Babersia microtia IRA, p32, p41,
viii. Bartonella henselae 17 kDa, 26 kDa, or SucB;
ix. Anaplasma phgocytophilum MSP5, MSP2, or OmpA;
x. Rickettsia typhi OmpB or surface antigen;
xi. Cytomeglovirus EIA, gB, p150, p28, p52, pp65, or p38;
xii. Epstein Barr virus EA, EBNA1, VCA, gp125, p18, or p23;
xiii. Parvovirus VLP VLP2, VLP VP1/VP2;
xiv. Toxoplasma gondii MIC3, p24, p29, or 30;
XV. Rickettsia rickettsia rompA or rompB;
xvi. Rickettsia parkeri OmpA, OmpB, PS 120, or 17 kDa;
xvii. Francisella tularensis LPS O antigen;
xviii. Heartland virus Gn or Gc; or
xix. lysate derived from one or more of Borrelia burgdorferi, Borrelia mayonii, Borrelia afzelii, Borrelia garinii, Borrelia Bavariensis, Borrelia speielmanii, Borrelia hemsii, Borrelia Turicatae, Borrelia miyamotoi, Borrelia andersonii, Borrelia maritima, Borrelia californiensis, Borrelia bissettiae, Borrelia lusitaniae, Borrelia valaisiana, Borrelia yangtzensis, Borrelia turcia, Babsia microti, Bartonella henselae, Anaplasma phagocytophilum, Ehrlichia chaffeensis, Rickettsia typhi, Rickettsia rickettsia, Powassan virus, Tick-borne encephalitis virus, West Nile virus, Coxsackie virus, Cytomegalovirus, Epstein Barr virus, Parvovirus B19, Toxoplasma, gondii, HSV-1, HSV-2, HHV-6, HHV-7, Colorado tick fever virus, Bourbon virus, or Borrelia lonestari.

In some embodiments, the two or more probe molecules comprise B. burgdorferi VLsE1, C6 peptide, B31, B297, p18, p28, p30, p31, p34, p39, P41, p45, p58, p66, and p93.

In some embodiments, the one or more tickborne pathogens is carried by at least one tick species selected from Table A.

In some embodiments, the microarray comprises two or more probe molecules from one or more tick species selected from Table A.

In some embodiments, the micro array comprises probe molecules from at least 1, 2, 3, 4, 5, 6, 7, 8, 9, 10, 11, 12, 13, 14, 15, 16, 17, 18, 19, 20, or more tick species.

In some embodiments, the tick species is any one of Ixodes scpularis, Ixodes ricinus, Ixodes persulatus, Ixodes uriae, Ornithodoros hermsi, Ornithodoros turicatae, Ixodes dentatus, Ixodes pacificus, Ixodes spinipalpis, Ixodes jellisonii, Ixodes nippopensis, Ixodes columnae, Ixodes granulatus, Hyalomma aegypticum; Amblyomma Americanum, Dermacentor variabilis, Dermacentor andersoni, Haemaphysalis longicornis, Ixodes cookie, Ornithodoros moubata, Amblyomma americanum, Dermacentor variabilis, Dermacentor andersoni, Rhipicephalus sanguineus, Dermacentor similis, Amblyomma Americanum, Amblyomma maculatum, Hemaphysalis longicornis, or Dermacentor similis.

In some embodiments, the tickborne pathogen generates an immune response in a subject.

In some embodiments, the tickborne pathogen causes a disease in a subject.

In some embodiments, the subject is human.

In some embodiments, the features are at positionally-defined locations on the surface.

In some embodiments, the surface comprises one or more silicon wafer microchips.

In some embodiments, the microarray comprises 10-100, 10-20, 20-30, 30-40, 40-50, 50-60, 60-70, 79-80, 80-90, 90-100, 100-150, 150-200, 200-250, 250-300, 300-350, 350-400, or more microchips, optionally 87 microchips.

In some embodiments, the microarray is attached to a support surface.

In some embodiments, the microchips are at positionally-defined locations on the support surface.

In some embodiments, the support surface is a pillar plate.

In some embodiments, the pillar plate comprises a 12, 24, 36, 48, or 96 pillar plate.

In one aspect, provided herein are methods for obtaining feature binding data, comprising:
i. obtaining the microarray disclosed herein,
ii. contacting the microarray with a sample comprising a plurality of ligands for at least a subset of the features under conditions that promote ligand binding; and
iii. imaging the microarray to identify binding of the plurality of ligands to the features of the microarray.

In one aspect, provided herein are methods of identifying a tickborne disease or pathogen in a subject, comprising:
i. contacting a sample from the subject with the microarray disclosed herein; and
ii. identifying binding of ligands in the sample to the features on the microarray to determine whether the subject has the tickborne disease or pathogen.

In some embodiments, the identifying binding of ligands in the sample comprises:
i. contacting the microarray with a sample comprising a plurality of ligands for at least a subset of the features under conditions that promote ligand binding; and
ii. imaging the microarray to identify binding of the plurality of ligands to the features of the microarray.

In some embodiments, the tickborne disease is Lyme disease, rickettsiosis, Rocky Mountain spotted fever, Southern tick-associated rash illness, tick-borne relapsing fever, tularemia, or Q fever.

In some embodiments, the pathogen is Borrelia burgdorferi, Borrelia mayonii, Borrelia afzelii, Borrelia garinii, Borrelia Bavariensis, Borrelia speielmanii, Borrelia hemsii, Borrelia Turicatae, Borrelia miyamotoi, Borrelia andersonii, Borrelia maritima, Borrelia californiensis, Borrelia bissettiae, Borrelia lusitaniae, Borrelia valaisiana, Borrelia yangtzensis, Borrelia turcia, Borrelia lonestari, Babsia microti, Babsia duncani, Bartonella henselae, Anaplasma phagocytophilum, Ehrlichia chaffeensis, Rickettsia typhi, Rickettsia rickettsia, Rickettsia parkeri, Francisella tularensis, Powassan virus, Tick-borne encephalitis virus, West Nile virus, Coxsackie virus, Cytomegalovirus, Epstein Barr virus, Parvovirus B19, Toxoplasma, gondii, Colorado tick fever virus, Heartland virus, Bourbon virus, HSV-1, HSV-2, HHV-6, or HHV-7.

In some embodiments, the sample is from a subject.

In some embodiments, the sample is blood or serum.

In some embodiments, the subject is human.

In some embodiments, the ligands comprise an antibody or antigen-binding fragment.

In some embodiments, the ligand comprises an IgG antibody or IgM antibody or a combination thereof.

In some embodiments, the method comprises identifying binding of IgG, IgM or IgG and IgM antibodies present in the sample to the probe molecules on the microarray.

In some embodiments, the method comprises a sensitivity of detection of IgM antibodies that bind to the tickborne disease or pathogen with a sensitivity of at least 25%, 55%, 60%, 65%, 70%, 75%, 80%, 85%, 90%, 91%, 92%, 93%, 94%, 95%, 96%, 97%, 98%, or 99%.

In some embodiments, the method comprises a specificity of detection of IgM antibodies that bind to the tickborne disease or pathogen with a sensitivity of at least 95%.

In some embodiments, the method comprises a sensitivity of detection of IgG antibodies that bind to the tickborne disease or pathogen with a sensitivity of at least 80%, 83%, 85%, 87%, 90%, 91%, 92%, 93%, 94%, 95%, 96%, 97%, 98%, 99%, or 100%.

In some embodiments, the method comprises a specificity of detection of IgM antibodies that bind to the tickborne disease or pathogen with a sensitivity of at least 95%, 96%, 97%, 98%, 99%, or 100%.

In some embodiments, the method comprises a sensitivity of detection of IgG and IgM antibodies that bind to the tickborne disease or pathogen with a sensitivity of at least 85%, 87%, 90%, 91%, 92%, 93%, 94%, 95%, 96%, 97%, 98%, 99%, or 100%.

In some embodiments, the method comprises a specificity of detection of IgG and IgM antibodies that bind to the tickborne disease or pathogen with a sensitivity of at least 95%, 96%, 97%, 98%, 99%, or 100%.

In some embodiments, a total number of features on the microarray is at least about 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 32, 330, 340, 350, 360, 370, 380, 390, or 400 or more.

In some embodiments, the microarray has an area that is less than or equal to 0.2 square millimeters (mm²), less than or equal to 0.7 square millimeters (mm²), less than or equal to 1 square millimeters (mm²), less than or equal to 10 square millimeters (mm²), less than or equal to 100 square millimeters (mm²), or less than or equal to 150 square millimeters (mm²).

In some embodiments, the sample has a volume that is less than or equal to 100 μL, less than or equal to 50 μL, less than or equal to 25 μL, less than or equal to 10 μL, less than or equal to 5 μL, less than or equal to 1.5 μL, or less than or equal to 1 μL.

In some embodiments, an elapsed time from sample contacting to finishing the imaging is equal or less than 20 minutes, equal or less than 5 minutes, equal or less than 1 minute, equal or less than 2-seconds, equal or less than 10 seconds, equal or less than 1 second.

In some embodiments, a coefficient of variation of data obtained from the array is not greater than 5 percent, not greater than 2 percent, not greater than 1 percent.

In some embodiments, the microarray comprises at least at least 1000 features per square centimeter, at least 5,000 features per square centimeter, at least 10,000 features per square centimeter, at least 50,000 features per square centimeter, at least 100,000 features per square centimeter, at least 500,000 features per square centimeter, at least 1,000,000 features per square centimeter, at least 10,000,000 features per square centimeter, or at least 15,000,000 features per square centimeter.

In some embodiments, the contacting occurs at a concentration of the plurality of ligands that is less or equal than 1,000 μg/ml in the sample, less or equal than 10 μg/ml in the sample, less or equal than 1 μg/ml in the sample, less or equal than 0.1 μg/ml in the sample, less or equal than 10 ng/ml in the sample, less or equal than 1 ng/ml in the sample, less or equal than 5 μg/ml in the sample.

In some embodiments, contacting occurs at a concentration of the plurality of ligands that is within the range of approximately 1 μg/ml to approximately 1,000 μg/ml in the sample.

In some embodiments, the imaging comprises identifying binding of at least 100 ligands, at least 500 ligands, at least 1,000 ligands, at least 100,000 ligands, at least 1,000,000 ligands, at least 10,000,000 ligands, at least 15,000,000 ligands, or at least 100,000,000 ligands to the features of the microarray.

### BRIEF DESCRIPTION OF THE SEVERAL VIEWS OF THE DRAWINGS

These and other features, aspects, and advantages of the present invention will become better understood with regard to the following description, and accompanying drawings, where:

FIG. 1 provides a schematic showing production and assembly of a microarray for diagnosis of tick pathogens.

FIG. 2 provides an exemplary microarray showing the location of different tickborne pathogen probe molecules on microchips assembled into a microarray.

FIG. 3 shows a lyme disease antigen reactivity heat map after using a chip with individual antigens of B. burgdorferi tested for reactivity with IgG and IgM antibodies. The indicated antigens are shown on the right.

### DETAILED DESCRIPTION

### Definitions

Terms used in the claims and specification are defined as set forth below unless otherwise specified.

As used herein the term “wafer” refers to a slice of semiconductor material, such as a silicon or a germanium crystal generally used in the fabrication of integrated circuits. Wafers can be in a variety of sizes from, e.g., 25.4 mm (1 inch) to 300 mm (11.8 inches) along one dimension with thickness from, e.g., 275 μm to 775 μm.

As used herein the term “photoresist” or “resist” or “photoactive material” refers to a light-sensitive material that changes its solubility in a solution when exposed to ultra violet or deep ultra violet radiation. Photoresists are organic or inorganic compounds that are typically divided into two types: positive resists and negative resists. A positive resist is a type of photoresist in which the portion of the photoresist that is exposed to light becomes soluble to the photoresist developer. The portion of the photoresist that is unexposed remains insoluble to the photoresist developer. A negative resist is a type of photoresist in which the portion of the photoresist that is exposed to light becomes insoluble to the photoresist developer. The unexposed portion of the photoresist is dissolved by the photoresist developer.

As used herein the term “photomask” or “reticle” or “mask” refers to an opaque plate with transparent patterns or holes that allow light to pass through. In a typical exposing process, the pattern on a photomask is transferred onto a photoresist.

As used herein the term “coupling molecule” includes in one embodiment any natural or artificially synthesized amino acid with its amino group protected with a fluorenylmethyloxycarbonyl (Fmoc) or tert-Butyloxycarbonyl (boc) group. These amino acids may optionally have their side chains protected. Examples of coupling molecules include, but are not limited to, boc-Gly-COOH, Fmoc-Trp-COOH. Other embodiments of coupling molecules include monomer molecules and combinations thereof that can form polymers upon coupling, e.g., nucleotides, sugars and the like, and are described below.

As used here in the term “coupling” or “coupling process” or “coupling step” refers to a process of forming a bond between two or more molecules such as a linker molecule or a coupling molecule. A bond can be a covalent bond such as a peptide bond. A peptide bond can a chemical bond formed between two molecules when the carboxyl group of one coupling molecule reacts with the amino group of the other coupling molecule, releasing a molecule of water (H₂O). This is a dehydration synthesis reaction (also known as a condensation reaction), and usually occurs between amino acids. The resulting —C(═O)NH-bond is called a peptide bond, and the resulting molecule is an amide.

As used herein the terms “polypeptide,” “peptide,” or “protein” are used interchangeably to describe a chain or polymer of amino acids that are linked together by bonds. Accordingly, the term “peptide” as used herein includes a dipeptide, tripeptide, oligopeptide, and polypeptide. The term “peptide” is not limited to any particular number of amino acids. In some embodiments, a peptide contains about 2 to about 50 amino acids, about 5 to about 40 amino acids, or about 5 to about 20 amino acids. A molecule, such as a protein or polypeptide, including an enzyme, can be a “native” or “wild-type” molecule, meaning that it occurs naturally in nature; or it may be a “mutant,” “variant,” “derivative,” or “modification,” meaning that it has been made, altered, derived, or is in some way different or changed from a native molecule or from another molecule such as a mutant. A “point mutation” refers to the mutation of one amino acid among the amino acids in a sequence of a peptide.

As used herein the term “biomarkers” includes, but is not limited to DNA, RNA, proteins (e.g., enzymes such as kinases), peptides, sugars, salts, fats, lipids, ions and the like.

As used herein the term “linker molecule” or “spacer molecule” includes any molecule that does not add any functionality to the resulting peptide but spaces and extends out the peptide from the substrate, thus increasing the distance between the substrate surface and the growing peptide. This generally reduces steric hindrance with the substrate for reactions involving the peptide (including uni-molecular folding reactions and multi-molecular binding reactions) and so improves performance of assays measuring one or more embodiments of peptide functionality.

As used herein the term “developer” refers to a solution that can selectively dissolve the materials that are either exposed or not exposed to light. Typically developers are water-based solutions with minute quantities of a base added. Examples include tetramethyl ammonium hydroxide in water-based developers. Developers are used for the initial pattern definition where a commercial photoresist is used. Use of developers is described in Example 1 below.

As used herein the term “protecting group” includes a group that is introduced into a molecule by chemical modification of a functional group in order to obtain chemoselectivity in a subsequent chemical reaction. “Chemoselectivity” refers to directing a chemical reaction along a desired path to obtain a pre-selected product as compared to another. For example, the use of boc as a protecting group enables chemoselectivity for peptide synthesis using a light mask and a photoacid generator to selectively remove the protecting group and direct pre-determined peptide coupling reactions to occur at locations defined by the light mask.

As used herein the term “microarray,” “chip” or “chip array” refers to a substrate on which a plurality of probe molecules of protein or specific DNA binding sequences have been affixed at separate locations in an ordered manner thus forming a microscopic array. Protein or specific DNA binding sequences may be bound to the substrate of the chip through one or more different types of linker molecules. A “chip array” refers to a plate having a plurality of chips, for example, 24, 96, or 384 chips.

As used herein the term “probe molecules” refers to, but is not limited to, proteins, peptides, (including recombinant proteins or peptides), antigens, antigenic fragments, DNA binding sequences, antibodies, oligonucleotides, nucleic acids, peptide nucleic acids (“PNA”), deoxyribonucleic acids (DNA), ribonucleic acids (RNA), peptide mimetics, nucleotide mimetics, chelates, biomarkers and the like. In some embodiments, “recombinant protein” or “recombinant peptide” refers to proteins or peptides that are produced by recombinant or synthetic DNA technology. In some embodiments, “protein” refers to a full length protein as opposed to a fragment. In some embodiments, “protein” refers to a chain of at least 50 amino acids. In some embodiments, a protein is a polypeptide. In some embodiments, “peptide” refers to a chain of two to 49 amino acids. In some embodiments, a peptide is a fragment of a protein.

As used herein, the term “feature” refers to a microchip or particular probe molecule that has been attached to a microarray in a positionally defined location.

As used herein, the term “ligand” refers to a molecule, agent, analyte or compound of interest that can bind to one or more features. For example, a ligand can be an antibody or antigen-binding fragment thereof, or an antigen binding protein. Exemplary antibodies include, but are not limited to, IgG and IgM antibodies.

As used herein the term “microarray system” or a “chip array system” refers to a system usually comprised of probe molecules formatted on a solid planar surface like glass, plastic or silicon chip plus the instruments needed to handle samples (automated robotics), to read the reporter molecules (scanners) and analyze the data (bioinformatic tools).

As used herein the term “patterned region” or “pattern” or “location” refers to a region on the substrate on which are grown different features. These patterns can be defined using photomasks.

As used herein the term “derivatization” refers to the process of chemically modifying a surface to make it suitable for bio molecular synthesis. Typically derivatization includes the following steps: making the substrate hydrophilic, adding an amino silane group, and attaching a linker molecule.

As used herein the term “capping” or “capping process” or “capping step” refers to the addition of a molecule that prevents the further reaction of the molecule to which it is attached. For example, to prevent the further formation of a peptide bond, the amino groups are typically capped by acetylation in the presence of an acetic anhydride molecule.

As used herein the term “diffusion” refers to the spread of, e.g., photoacid through random (i.e., Brownian) motion from regions of higher concentration to regions of lower concentration.

As used herein the term “dye molecule” refers to a dye which typically is a colored substance that can bind to a substrate. Dye molecules can be useful in detecting binding between a feature on an array and a ligand, e.g. a molecule of interest.

As used herein, the terms “immunological binding” and “immunological binding properties” refer to the non-covalent interactions of the type which occur between an immunoglobulin molecule and an antigen for which the immunoglobulin is specific.

As used herein the term “biological sample” refers to a sample derived from biological tissue or fluid that can be assayed for an analyte(s) of interest or any ligand. Such samples include, but are not limited to, sputum, amniotic fluid, blood, blood cells (e.g., white cells), tissue or fine needle biopsy samples, urine, peritoneal fluid, and pleural fluid, or cells therefrom. Biological samples may also include sections of tissues such as frozen sections taken for histological purposes. Although the sample is typically taken from a human patient, the assays can be used to detect analyte(s) of interest in samples from any organism (e.g., mammal, bacteria, virus, algae, or yeast) or mammal, such as dogs, cats, sheep, cattle, and pigs. The sample may be pretreated as necessary by dilution in an appropriate buffer solution or concentrated, if desired.

As used herein, the term “assay” refers to a type of biochemical test that measures the presence or concentration of a substance of interest in solutions that can contain a complex mixture of substances.

The term “antigen” as used herein refers to a molecule that triggers an immune response by the immune system of a subject, e.g., the production of an antibody by the immune system. Antigens can be exogenous, endogenous or auto antigens. Exogenous antigens are those that have entered the body from outside through inhalation, ingestion or injection. Endogenous antigens are those that have been generated within previously-normal cells as a result of normal cell metabolism, or because of viral or intracellular bacterial infection. Auto antigens are those that are normal protein or protein complex present in the host body but can stimulate an immune response. In some embodiments, antigens include whole proteins that give rise to an immune response and fragments of those proteins that include the antigenic portion (i.e., “antigenic fragments”). In some embodiments, the antigen or antigenic fragment is a recombinant protein or peptide.

As used herein the term “epitope” or “immunoactive regions” refers to distinct molecular surface features of an antigen capable of being bound by component of the adaptive immune system, e.g., an antibody or T cell receptor. Antigenic molecules can present several surface features that can act as points of interaction for specific antibodies. Any such distinct molecular feature can constitute an epitope. Therefore, antigens have the potential to be bound by several distinct antibodies, each of which is specific to a particular epitope.

As used herein the term “antibody” or “immunoglobulin molecule” refers to a molecule naturally secreted by a particular type of cells of the immune system: B cells. There are five different, naturally occurring isotypes of antibodies, namely: IgA, IgM, IgG, IgD, and IgE.

The term percent “identity,” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information website.

It must be noted that, as used in the specification and the appended claims, the singular forms “a,” “an,” and “the” include plural referents unless the context clearly dictates otherwise.

### Compositions

### Microarrays and Arrays

The microarray design disclosed herein includes probe molecules (e.g. protein, peptides, lysates, or antigens, or any other probe molecule disclosed herein) from tickborne pathogens separated by design and can multiplex across species. It can also multiplex across antigen types such as recombinant proteins, peptides, and lysates simultaneously. This method can lower test costs since all the infections can be detected simultaneously using a single run. The multiplex chip microarray has three main advantages over the existing technologies. First, it has an ultra-high-density microarray surface with high reproducibility and better throughput. Second, it can detect a large number of antibodies against various infectious agents at the same time. Third, detection of antibodies can be performed using low sample volumes with low cost and a fast turnaround time. Given the flexible nature of the multiplex platform, the chip microarray can provide a multiplexed testing solution for Lyme disease, the B. burgdorferi spirochete, other tick pathogen co-infections, and other infections of interest.

Also disclosed herein are microarrays comprising one or more microchips comprising features (e.g., probe molecules) attached to the surface of the one or more microchips and wherein the features comprise one or more probe molecule from one or more tickborne pathogens. Some embodiments of a microarray comprise one or more microchips, each microchip comprising a substrate and features attached to the substrate. The microchips can be assembled on the planar surface of a pillar plate to form a customizable microarray of selected probe molecules. Other embodiments of an microarray comprise a substrate and features attached to the substrate surface at positionally-defined locations. In some embodiments, the substrate surface can be the planar surface of a pillar plate.

In some embodiments, the features attached to the substrate surface are selected from a group consisting of: proteins, peptides, antigens, antigenic fragments, DNA binding sequences, antibodies, oligonucleotides, nucleic acids, peptide nucleic acids, deoxyribonucleic acids, ribonucleic acids, peptide mimetics, nucleotide mimetics, chelates, biomarkers, and the like.

In some embodiments, the substrate surface of the microarray (such as the substrate surface of a microchip or pillar plate) is functionalized with free amine or free carboxylic acids for polypeptide synthesis. In some embodiments, the free carboxylic acids are activated to bind to amine groups, e.g., during polypeptide synthesis on the surface of the microarray.

In some embodiments, a microarray comprises two-dimensional array, wherein the features occupy a 2-dimensional plane. In some embodiments, a microarray comprises two-dimensional array, wherein the positionally-defined locations occupy a 2-dimensional plane. For example, each feature can comprise: a probe molecule or a collection of protein or peptide chains of determinable sequence and intended length, wherein within an individual feature, the fraction of peptide chains within said collection having the intended length is characterized by an average coupling efficiency for each coupling step of about 98%. In some embodiments, the average coupling efficiency for each coupling step is at least 98.5%. In some embodiments, the average coupling efficiency for each coupling step is at least 99%. In some embodiments, the average coupling efficiency for each coupling step is at least 90, 91, 92, 93, 94, 95, 96, 97, 98, 98.5, 98.6, 98.7, 98.8, 98.9, 99.0, 99.1, 99.2, 99.3, 99.4, 99.5, 99.6, 99.7, 99.8, 99.9, or 100%.

In some embodiments, the surface density of features on the microarray is greater than 10/cm², 100/cm², 1,000/cm², 10,000/cm², 100,000/cm², 1,000,000/cm², 10,000,000/cm² or 20,000,000/cm². In some embodiments, the total number of features on the microarray or array is at least about 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 32, 330, 340, 350, 360, 370, 380, 390, 400, 500, 600, 700, 800, 900, 1,000, 1100, 1200, 1300, 1400, 1500, 1600, 1700, 1800, 1900, 2000, 3000, 4000, 5,000, 6000, 7000, 8000, 9,000, 10,000 11,000, 12,000, 13,000, 14,000, 15,000, 16,000, 17,000, 18,000, 19,000, 20,000, 30,000, 40,000, 50,000, 60,000, 70,000, 80,000, 90,000, 100,000, 200,000, 300,000, 400,000, 500,000, 600,000, 700,000, 800,000, 900,000, 1,000,000, 2,000,000, 3,000,000, 4,000,000, 5,000,000, 6,000,000, 7,000,000, 8,000,000, 10,000,000, 12,000,000, 14,000,000, 16,000,000, or 18,000,000, or, between about 2-10, 10-20, 20-30, 30-40, 40-50, 50-60, 60-70, 70-80, 80-90, 90-100, 110-120, 120-130, 130-140, 140-150, 150-160, 160-170, 170-180, 180-190, 190-200, 210-220, 220-230, 230-240, 240-250, 250-260, 260-270, 270-280, 280-290, 290-300, 310-320, 320-330, 330-330, 340-350, 350-360, 360-370, 370-380, 380-390, 390-400, 400-500, 500-600, 600-700, 700-800, 800-900, 900-1000, or more. In other embodiments, the size of the microarray is less than or equal to 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 200, 250, 300, 400, 500, 600, 700, 800, 900, or 1,000 square millimeters.

In some embodiments, the microarray is comprised of at least one microchip (e.g., a plurality microchips) to which the features are attached. The collection of microchips is the microarray. In such embodiments, the size of each microchip in the microarray can be from 0.5 mm to 2 mm. In some embodiments, the microarray comprises 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 32, 330, 340, 350, 360, 370, 380, 390, 400, or more microchips, optionally 87 microchips. In such embodiments, a distinct probe molecule (e.g., a tick pathogen disclosed herein) is coupled to each individual microchip and the microchips are assembled on a surface (e.g., a surface of a pillar on a pillar plate) to create the microarray.

In some embodiments, a microarray can be a three-dimensional array, e.g., the substrate comprising a porous layer with features attached to the surface of the porous layer. In some embodiments, the surface of a porous layer includes external surfaces and surfaces defining pore volume within the porous layer. In some embodiments, a three-dimensional microarray can include features attached to a surface at positionally-defined locations, said features each comprising: a collection of peptide chains of determinable sequence and intended length. In one embodiment, within an individual feature, the fraction of peptide chains within said collection having the intended length is characterized by an average coupling efficiency for each coupling step of greater than 98%. In some embodiments, the average coupling efficiency for each coupling step is at least 90, 91, 92, 93, 94, 95, 96, 97, 98, 98.5, 98.6, 98.7, 98.8, 98.9, 99.0, 99.1, 99.2, 99.3, 99.4, 99.5, 99.6, 99.7, 99.8, 99.9, or 100%.

In some embodiments, each peptide chain is from 5 to 60 amino acids in length. In some embodiments, each peptide chain is at least 5 amino acids in length. In some embodiments, each peptide chain is at least 5, 10, 15, 20, 25, 30, 35, 40, 45, 50, 55, or 60 amino acids in length. In some embodiments, each peptide chain is less than 5, at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, or greater than 60 amino acids in length. In some embodiments, each peptide chain comprises one or more L amino acids. In some embodiments, each peptide chain comprises one or more D amino acids. In some embodiments, each peptide chain comprises one or more naturally occurring amino acids. In some embodiments, each peptide chain comprises one or more synthetic amino acids.

In some embodiments, a microarray can include at least 100, 200, 300, 400, 500, 600, 700, 800, 900, or 1,000 different probe molecules such as lysates, proteins, or peptide chains attached to the surface. In some embodiments, a microarray can include at least 10,000 different probe molecules such as lysates, proteins, or peptide chains attached to the surface. In some embodiments, a microarray can include at least 100, 200, 300, 400, 500, 600, 700, 800, 900, 1000, 2000, 3000, 4000, 5000, 6000, 7000, 8000, 9000, 10,000, or greater than 10,000 different probe molecules such as lysates, proteins, or peptides attached to the surface (or any integer in between).

In some embodiments, a microarray can include a single protein, peptide chain, lysate, protein, or antibody attached to a plurality of different types of linker molecules. In some embodiments a microarray can include at least 2 different types of linker molecules. In some embodiments, a microarray can include at least 2, 3, 4, 5, 6, 7, 8, 9, 10, 15, 20, 25, 30, 40, 50, 75, or greater than 100 different types of linker molecules attached to the substrate.

In some embodiments, each of the positionally-defined locations is at a different, known location that is physically separated from each of the other positionally-defined locations. In some embodiments, each of the positionally-defined locations is a positionally-distinguishable location. In some embodiments, each determinable sequence is a known sequence. In some embodiments, each determinable sequence is a distinct sequence.

In some embodiments, the features are covalently attached to the surface. In some embodiments, said peptide chains are attached to the surface through a linker molecule or a coupling molecule.

In some embodiments, the features comprise a plurality of distinct, nested, overlapping peptide chains comprising subsequences derived from a source protein having a known sequence. In some embodiments, each peptide chain in the plurality is substantially the same length. In some embodiments, each peptide chain in the plurality is the same length. In some embodiments, each peptide chain in the plurality is at least 5 amino acids in length. In some embodiments, each peptide chain in the plurality is at least 5, 10, 15, 20, 25, 30, 35, 40, 45, 50, 55, or 60 amino acids in length. In some embodiments, each peptide chain in the plurality is less than 5, at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, or greater than 60 amino acids in length. In some embodiments, at least one peptide chain in the plurality is at least 5 amino acids in length. In some embodiments, at least one peptide chain in the plurality is at least 5, 10, 15, 20, 25, 30, 35, 40, 45, 50, 55, or 60 amino acids in length. In some embodiments, at least one peptide chain in the plurality is less than 5, at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, or greater than 60 amino acids in length. In some embodiments, each polypeptide in a feature is substantially the same length. In some embodiments, each polypeptide in a feature is the same length. In some embodiments, the features comprise a plurality of peptide chains each having a random, determinable sequence of amino acids.

Sensitivity (true positive rate) is the probability of a positive test result, conditioned on the sample truly being positive. Sensitivity refers to a test's ability to diagnosis a sample as positive. A highly sensitive test means that there are few false negative results. Specificity (true negative rate) is the probability of a negative test result, conditioned on the sample truly being negative. The specificity of a test is its ability to diagnosis a sample as negative.

In some embodiments, the microarray is configured to have an at least 25%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% sensitivity for detection of a tickborne pathogen after contact of the features or probe molecules with a sample from a subject suspected of having a tickborne pathogen.

In some embodiments, the microarray is configured to have an at least 80%, 81%, 82%, 83%, 84%, 85%, 86%, 87%, 88%, 89%, 90%, 91%, 92%, 93%, 94%, 95%, 96%, 97%, 98%, 99%, or 100% specificity for detection of a tickborne pathogen after contact of the features or probe molecules with a sample from a subject suspected of having a tickborne pathogen.

In some embodiments, the microarray is configured to have an at least 25%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% sensitivity and an at least 80%, 81%, 82%, 83%, 84%, 85%, 86%, 87%, 88%, 89%, 90%, 91%, 92%, 93%, 94%, 95%, 96%, 97%, 98%, 99%, or 100% specificity for detection of a tickborne pathogen after contact of the features or probe molecules with a sample from a subject suspected of having a tickborne pathogen.

In some embodiments, the microarray is configured to have at least 90% sensitivity and 90% specificity for detection of a tickborne pathogen after contact of the features or probe molecules with a sample from a subject suspected of having a tickborne pathogen.

In some embodiments, the microarray is configured to have at least 93% sensitivity and 96% specificity for detection of a tickborne pathogen after contact of the features or probe molecules with a sample from a subject suspected of having a tickborne pathogen.

In some embodiments, the microarray is configured to have at least 95% sensitivity and 95% specificity for detection of a tickborne pathogen after contact of the features or probe molecules with a sample from a subject suspected of having a tickborne pathogen.

Microarray manufacturing, analyzing, and imaging is also described in U.S. Pat. Nos. 9,417,236; 10,175,234, 10,746,732, 11,674,956, and 10,006,909, each of which are hereby incorporated by reference in their entirety.

### Chip Arrays

Also disclosed herein are chip arrays comprising one or more microarrays (also called “chip array”). In some embodiments, a chip array is a two-dimensional array of microarrays on a support layer or plate. In some embodiments, the support layer or plate is a pillar plate. In some embodiments of chip arrays, each chip array only comprises a single protein, peptide, lysate or sonicate, or antibody microarray. In other embodiments, each chip array comprises a plurality of microarrays comprising proteins, antibodies, peptides, lysates (e.g. whole cell lysate or sonicate), oligonucleotides, DNA, RNA, peptide nucleic acid (“PNA”), probe molecules and the like. In some embodiments, microarrays are packaged (e.g., attached) onto a 96 well or pillar plate. In some embodiments, microarrays are packaged onto a 24 well or pillar plate. In some embodiments, microarrays are packaged onto a 36 well or pillar plate. In some embodiments, microarrays are packaged onto a 48 well or pillar plate. In some embodiments, microarrays are packaged onto a 12 well or pillar plate. In some embodiments, epoxy is used to attach a microarray comprising at least one microchip to the wafer. In some embodiments, the support layer is an array of pillars, and a chip or a plurality of chips (e.g., microchips) is attached to each pillar. These pillars of the support layer are of macroscopic scale and are to be distinguished from the substrate pillars described above. In other embodiments, a chip (e.g., microchips) is attached to a cap which attaches to a pillar on a pillar plate.

In one embodiment, chips (e.g., microchips) are formed on a silicon wafer, the silicon wafer being the surface, and then diced into multiple chips of varying dimensions. In some embodiments, each chip (e.g., microchips) has a dimension of 0.1 mm by 0.1 mm up to 2 cm to 2 cm. In some embodiments, each chip (e.g., microchips) has a dimension of 0.7 mm by 0.7 mm. In some embodiments, each chip (e.g., microchips) has a dimension of 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, or 2 mm². In some embodiments, the chips formed on a wafer and diced into multiple chips fit onto 12-, 24-, 36-, 48-, 96-, 192-, or 384-well plates, or any other custom made plates. In some embodiments, these plates have a plurality of wells which act as containers for each chip. In some embodiments, the plate is used for in-vitro diagnostics, such as protein-protein interaction assays or other enzymatic reactions. The microchips can then be assembled on a support surface, such as a pillar plate, to form the microarray of features comprising probe molecules.

### Robotic Chip Array System

In some embodiments, the assay station is automated to perform liquid handling on the chip array. In some embodiments, the liquid handling assay station is any commercially available one that can use the standard or custom made well plates which hold the plurality of chips. After performing the assay using a liquid handling assay station, the chip is scanned using any commercially available confocal or CCD scanner. In some embodiments, the confocal scanner scans multiple chips loaded onto the substrate. In some embodiments, the data from the confocal scanner is analyzed on a Vibrant Bio Analyzer.

In some embodiments, one or several autoloader units feed the plate to the liquid handling assay station. Once the assay is performed, the chips are scanned on the confocal scanner using an autoloader. In some embodiments, one or several confocal scanners are connected to the autoloader to allow the autoloader to transfer chip arrays to a one or a plurality of scanners.

The packaging process of the chips including the steps of: dicing the quality controlled processed wafer into chips, picking the diced chips from the diced wafer and placing them onto a tape, picking the chips from the tape and attaching them onto a pillar plate using adhesive, and storing the pillar plates with chips attached to each pillar for future use.

The bioassay process of the pillar plates with chips attached to each plate including the steps of: placing and washing the pillar plate in a first well plate filled methanol, picking up the pillar plate from the first well plate and transporting it to a second well plate filled with TBS for washing. In the third step, the process places the pillar plate in third well plate for incubation with the primary antibody, followed by washing the pillar plate in a fourth well plate containing PBST. The next step includes placing the pillar plate in a fifth well plate for incubation with the secondary antibody, followed by washing the pillar plate in a sixth well plate with PBST and then by washing it in a seventh well plate with DI water before drying the pillar plate in nitrogen for further analysis.

The scanning process of the assayed chips including the steps of: checking the chips under the microscope to determine if they are clean and ready for scanning, washing the chips in DI water if the chips are determined to be contaminated, scanning the chips by using a confocal scanner microscope to determine the signal intensity for each feature located on the chips.

### Tick Species and Pathogens

In some aspects, the features and probe molecules are derived from tickborne pathogens. Such pathogens can be bacterial or viral pathogens carried by any tick species. In some embodiments, a tickborne pathogen is a bacteria or virus that causes a disease in a mammal host, such as a human. Such tickborne pathogens include, but are not limited to, Borrelia burgdorferi, Borrelia mayonii, Borrelia afzelii, Borrelia garinii, Borrelia Bavariensis, Borrelia speielmanii, Borrelia hemsii, Borrelia Turicatae, Borrelia miyamotoi, Borrelia andersonii, Borrelia maritima, Borrelia californiensis, Borrelia bissettiae, Borrelia lusitaniae, Borrelia valaisiana, Borrelia yangtzensis, Borrelia turcia, Borrelia lonestari, Babsia microti, Babsia duncani, Bartonella henselae, Anaplasma phagocytophilum, Ehrlichia chaffeensis, Rickettsia typhi, Rickettsia rickettsia, Rickettsia parkeri, Francisella tularensis, Powassan virus, Tick-borne encephalitis virus, West Nile virus, Coxsackie virus, Cytomegalovirus, Epstein Barr virus, Parvovirus B19, Toxoplasma, gondii, Colorado tick fever virus, Heartland virus, Bourbon virus, HSV-1, HSV-2, HHV-6, or HHV-7. In some embodiments, the tickborne pathogen is associated with Lyme disease, rickettsiosis, Rocky Mountain spotted fever, Southern tick-associated rash illness, tick-borne relapsing fever, tularemia, or Q fever. In some embodiments, the tickborne pathogen causes Lyme disease, rickettsiosis, Rocky Mountain spotted fever, Southern tick-associated rash illness, tick-borne relapsing fever, tularemia, or Q fever. Tick pathogens are available from a variety of vendors including ATCC, DSMZ, ZeptoMetrix, and Biospacific. See Table 2 for strains, vendor info, and catalogue numbers.

Any molecule derived from a tickborne pathogen can be used as the probe molecule on the microarray described here. Such molecules include, but are not limited to, lysates, antigens or antigenic fragments, proteins, or peptides. The probe molecules such as antigens or antigenic fragments, proteins, or peptides can be isolated from the pathogens or host tick species, or produced by recombinant or synthetic DNA technologies.

Lysates, such as whole cell lysates, can be prepared by mechanical, physical, chemical, or sonic disruption of cells. Whole cell lysate includes whole cell sonicate preparations made by sonicating cells to lyse them. Additional methods of preparing whole cell lysates include chemical disruption with detergents or chaotropic agents, alkaline agents such as sodium hydroxide, or enzymes, such as cellulose, protease, lysozyme, zymolyase, lysostaphin, or glycanase; mechanical disruption such as high-pressure homogenizers or bead mills; physical disruption using heat, pressure, sonic (sound) energy, cavitation, thermolysis (freeze-thaw cycles), or osmotic shock.

In some embodiments, the features comprising probe molecules comprise one or more of a lysate, antigens or antigenic fragments, proteins, or peptides. In some embodiments, the features comprising probe molecules comprise at least one lysate, at least one protein, and at least one peptide. In some embodiments, the features comprising probe molecules comprise the same type of probe molecules, e.g., only lysates or only peptides. In some embodiments, the probe molecules comprise different types of probe molecules, e.g., a lysate and a peptide, or a peptide and a protein, or a lysate, a peptide, and a protein.

Any combination of probe molecules derived from a tickborne pathogen can be used on the microarray. For example, the microarray can comprise one, two three, four, etc, or more of: B. burgdorferi VLsE1, C6 peptide, DbpB, OspC, p18, p32, p28, p30, p31, OspA, OspB, BmpA, p34, p39, P41, p45, p58, p66, p83, p84, p85 p86, p87, p88, p89, p90, p91, p92, p93, whole cell sonicate crude extract B31, or whole cell sonicate crude extract B297; B. afzelii BmpA, DbpA, OspA, OscpC, or p100; B. garinii DBpA or OspC; B. bavariensis p58, VLsE1, DbpA; B. spielmanii DBpA or OspC; B. miyamotoi GlpQ; Babersia microtia IRA, p32, p41, Bartonella henselae 17 kDa, 26 kDa, or SucB; Anaplasma phgocytophilum MSP5, MSP2, or OmpA; Rickettsia typhi OmpB or surface antigen; Cytomeglovirus EIA, gB, p150, p28, p52, pp65, or p38; Epstein Barr virus EA, EBNA1, VCA, gp125, p18, or p23; Parvovirus VLP VLP2, VLP VP1/VP2; Toxoplasma gondii MIC3, p24, p29, or 30; Rickettsia rickettsia rompA or rompB; Rickettsia parkeri OmpA, OmpB, PS 120, or 17 kDa; Francisella tularensis LPS O antigen; Heartland virus Gn or Gc or whole cell sonicate derived from one or more of Borrelia burgdorferi, Borrelia mayonii, Borrelia afzelii, Borrelia garinii, Borrelia Bavariensis, Borrelia speielmanii, Borrelia hemsii, Borrelia Turicatae, Borrelia miyamotoi, Borrelia andersonii, Borrelia maritima, Borrelia californiensis, Borrelia bissettiae, Borrelia lusitaniae, Borrelia valaisiana, Borrelia yangtzensis, Borrelia turcia, Babsia microti, Bartonella henselae, Anaplasma phagocytophilum, Ehrlichia chaffeensis, Rickettsia typhi, Powassan virus, Tick-borne encephalitis virus, West Nile virus, Coxsackie virus, Cytomegalovirus, Epstein Barr virus, Parvovirus B19, Toxoplasma, gondii, HSV-1, HHV-6, Colorado tick fever virus, Bourbon virus, or Borrelia lonestari. Amino acid sequences of the specific probe molecules derived from a tickborne pathogen listed herein can be identified via the UniProt database as of Sep. 8, 2023. UniProt identifiers for exemplary pathogen proteins are provided in Table B.

In some embodiments, the one or more (e.g., one, two, three, four, five, etc) comprise Borrelia burgdorferi VLsE1, C6 peptide, DbpB, OspC, OspA, OspB, BmpA, B31, B297, p18, p28, p30, p31, p34, p39, P41, p45, p58, p66, p83-93, WCS B31, and/or WCS 297 antigens. In some embodiments, the one or more (e.g., one, two, three, four, five, etc) comprise Borrelia afzelii BmpA, DbpA, OspA, OspC, and/or p100 antigens. In some embodiments, the one or more (e.g., one, two, three, four, five, etc) comprise Borrelia garinii DBpA and/or OspC antigens. In some embodiments, the one or more (e.g., one, two, three, four, five, etc) comprise Borrelia bavariensis p58, VLsE1, and/or DbpA antigens. In some embodiments, the one or more (e.g., one, two, three, four, five, etc) comprise Borrelia spielmanii DBpA and/or OspC antigens. In some embodiments, the one or more (e.g., one, two, three, four, five, etc) comprise Borrelia miyamotoi GlpQ antigen. In some embodiments, the one or more (e.g., one, two, three, four, five, etc) comprise Babesia microti IRA, p32, p41, and/or WCS antigens. In some embodiments, the one or more (e.g., one, two, three, four, five, etc) comprise Bartonella henselae 17 kDa, 26 kDa, and/or SucB antigens. In some embodiments, the one or more (e.g., one, two, three, four, five, etc) comprise Rickettsia typhi Omp B and/or surface antigen antigens. In some embodiments, the one or more (e.g., one, two, three, four, five, etc) comprise Anaplasma phagocytophilum MSP5, MSP2, and/or OmpA antigens. In some embodiments, the one or more (e.g., one, two, three, four, five, etc) comprise Cytomegalovirus EIA, gB, p150, p28, p52, pp65, and/or p38 antigens. In some embodiments, the one or more (e.g., one, two, three, four, five, etc) comprise Epstein Barr virus EA, EBNA1, VCA gp125, p18, and/or p23 antigens. In some embodiments, the one or more (e.g., one, two, three, four, five, etc) comprise Parvovirus VLP VLP2 and/or VLP VP1/VP2 Co Capsid antigens. In some embodiments, the one or more (e.g., one, two, three, four, five, etc) comprise Toxoplasma gondii WCS, MIC3, p24, p29, and/or p30 antigens.

In some embodiments, the one or more (e.g., one, two, three, four, five, etc) probe molecules comprise Anaplasma Phagocytophilum Msp5, Anaplasma Phagocytophilum p44, Borreliella afzelii DbpA, Borreliella afzelii OspA, Borreliella afzelii OspC, Borreliella burgdorferi DbpB, Borreliella burgdorferi OspA, Borreliella burgdorferi OspC, Borreliella burgdorferi p30, Borreliella burgdorferi p41, Borreliella burgdorferi p66, Borreliella garinii DbpA, Borreliella garinii OspC, B. burgdorferi p100, Parvovirus B19 VLP VP2, Parvovirus B19 VLP VP1/Vp2 Co Capsid, Borrelia mayonii nagB, Borrelia hermsii vsp13, Borrelia coriaceae lon, Babesia duncani coxIII, Bartonella elizabethae pth, Bartonella vinsonii virB3, Borrelia maritima YjgP/YjgQ, Borrelia californiensis S1, Borrelia bissettiae bptA, Borrelia valaisiana lipoprotein, Borrelia turcica enolase, Bartonella quintana hbpA, Toxoplasma gondii MIC3, Toxoplasma gondii P24, Toxoplasma gondii p30, Powassan Virus polyprotein, West Nile Virus NS1, Coxackievirus VP1, Tick-Borne Encephalitis Virus NS1 Protein, Parvovirus PepC (VP1 N-term), Borrelia burgdorferi VIsE1, OSpB Controll, Recombinant Rickettsia rickettsii (omp), Rickettsia typhi OmpB, Borrelia miyamotoi GIpQ, Chlamydia Pneumonia Recombinant, Streptolysin O, HHV Strain 6B, HHV Strain 6A, HSV1 gG1, HSV2 gG2, Cytomegalovirus antigen pp150, CMV Glycoprotein B, Antigen EBNA1, Antigen 23, and/or Cytomegalovirus pp65 protein.

In some embodiments, the one or more probe molecules is selected from Table B, Table 3, or Table 4.

Any pathogen carried by any tick species can be used to create the probe molecules on the microarray. In some embodiments, the one or more tickborne pathogens is carried by at least one tick species selected from Table 2 or Table 3. Pathogens from multiple species can be combined on the same microarray. In some embodiments, the one or more tickborne pathogens is carried by multiple tick species selected from Table 2 or Table 3. In some embodiments, the one or more tickborne pathogens is carried by at least one tick species selected from Table 2 or Table 3. In some embodiments, the microarray probe molecules are derived from at least 1, 2, 3, 4, 5, 6, 7, 8, 9, 10, 11, 12, 13, 14, 15, 16, 17, 18, 19, 20, or more tick species. In some embodiments, the at least one tick species is any one of Ixodes scpularis, Ixodes ricinus, Ixodes persulatus, Ixodes uriae, Ornithodoros hermsi, Ornithodoros turicatae, Ixodes dentatus, Ixodes pacificus, Ixodes spinipalpis, Ixodes jellisonii, Ixodes nippopensis, Ixodes columnae, Ixodes granulatus, Hyalomma aegypticum; Amblyomma Americanum, Dermacentor variabilis, Dermacentor andersoni, Haemaphysalis longicornis, Ixodes cookie, Ornithodoros moubata, Amblyomma americanum, Dermacentor variabilis, Dermacentor andersoni, Rhipicephalus sanguineus, Dermacentor similis, Amblyomma Americanum, Amblyomma maculatum, Hemaphysalis longicornis, or Dermacentor similis.

In some embodiments, the tickborne pathogen generates an immune response in a subject. In some embodiments, the tickborne pathogen causes a disease in a subject. In some embodiments, the disease is Lyme disease.

Table A provides a list of exemplary tickborne pathogens that can be analyzed by the microarrays disclosed herein, the tick that transmits each exemplary pathogen, the endemic region of the ticks, and the current diagnostic tests available. In some embodiments, the tickborne pathogen is selected from Table A. In some embodiments, the tick species is selected from Table A.

TABLE A

| Pathogen | Mode of transmission | Endemic Region | Gold std. Diagnostic test |
|---|---|---|---|
| Borrelia | Ixodes scapularis | New England, the | Two-tiered serological testing: |
| burgdorferi | | Mid-Atlantic states, | EIA or IFA followed by |
| | | Wisconsin, and | western blot. |
| | | Minnesota | |
| Borrelia mayonii | Ixodes scapularis | Upper Midwest | Not well established. Two-tier |
| | | | serological testing can be done. |
| Borrelia afzelii | Ixodes ricinus, Ixodes | Europe | Two-tiered serological testing: |
| | persulatus | | EIA or IFA followed by |
| | | | western blot. |
| Borrelia garinii | Ixodes ricinus, Ixodes | Europe and Asia | Two-tiered serological testing: |
| | persulatus | | EIA or IFA followed by |
| | | | western blot. |
| Borrelia | Ixodes uriae, Ixodes | Europe and Asia | Not yet established |
| bavariensis | persulcatus | | |
| Borrelia spielmanii | Ixodes ricinus | Europe | Not yet established. Two-tiered |
| | | | testing may be effective. |
| Borrelia hermsii | Ornithodoros hermsi | Western USA and | Not well established. |
| | | Canada | |
| Borrelia turicatae | Ornithodoros turicatae | USA and Mexico | Not yet established. Two-tiered |
| | | | testing may be effective. |
| Borrelia miyamotoi | Ixodes dentatus, Ixodes | Japan, USA, Sweden | Not well established. |
| | ricinus, Ixodes scapularis, | | |
| | Ixodes pacificus | | |
| Borrelia | Ixodes dentatus | USA | Not well-established |
| andersonii | | | |
| Borrelia maritima | Ixodes spinipalpis | San Francisco Bay | Not well established. |
| | | area, CA | |
| Borrelia | Ixodes jellisonii, Ixodes | California | Not well established |
| californiensis | spinipalpis, Ixodes | | |
| | pacificus | | |
| Borrelia bissettiae | I. scapularis, Ixodes | Asia and Europe | Not well established |
| | persulatus, Ixodes. | | |
| | spinipalpis, Ixodes | | |
| | pacificus | | |
| Borrelia lusitaniae | Ixodes ricinus | Europe and Northern | Not well established |
| | | Africa | |
| Borrelia valaisiana | Ixodes ricinus, Ixodes | Europe, Asia | Not well established |
| | nippopensis, Ixodes | | |
| | columnae | | |
| Borrelia | Ixodes granulatus, Ixodes | Asia | Not well established |
| yangtzensis | nipponensis | | |
| Borrelia. turcica | Hyalomma aegypticum | Several Southeastern | Not well established |
| | | European countries | |
| | | such as Greece, | |
| | | Bulgaria, Romania | |
| | | and Croatia | |
| Babesia microti | Ixodes ricinus, Ixodes | Northeastern and | Examination of the blood |
| | scapularis, blood | upper Midwestern | smear. |
| | transfusions, perinatal | United States | |
| Babesia duncani | Ixodes ricinus, Ixodes | Washington and | Not well established. Usually |
| | scapularis, blood | California | diagnosed using clinical |
| | transfusions, perinatal | | presentation, laboratory testing, |
| | | | and a history of tick exposure. |
| Bartonella | Bite or scratch wounds of | Worldwide | Bacterial culture, molecular- |
| henselae | dogs and cats, blood | | based detection (mostly used), |
| | transmission, mother- | | IFA, western blot |
| | fetus transmission, flies | | |
| Bartonella | Bite or scratch wounds of | US and Sweden | Bacterial culture, molecular- |
| elizabethae | dogs and cats, blood | (intravenous drug | based detection (mostly used), |
| | transmission, mother- | users) | IFA, western blot |
| | fetus transmission, flies | | |
| Bartonella | Bite or scratch wounds of | | Bacterial culture, molecular- |
| vinsonii | dogs and cats, blood | | based detection (mostly used), |
| | transmission, mother- | | IFA, western blot |
| | fetus transmission, flies | | |
| Bartonella | Bite or scratch wounds of | Worldwide | Bacterial culture, molecular- |
| quintana | dogs and cats, blood | | based detection (mostly used), |
| | transmission, mother- | | IFA, western blot |
| | fetus transmission, flies | | |
| Anaplasma | Ixodes scapularis, Ixodes | Northeast United | PCR |
| phagocytophilum | ricinus | States, northern | |
| | | Europe, and | |
| | | Southeast Asia | |
| Ehrlichia | Amblyomma americanum | Southcentral and | PCR |
| chaffeensis | | eastern U.S | |
| Rickettsia typhi | Tick (Dermacentor | Africa, Asia, and the | Serological testing like ELISA, |
| | variabilis, Dermacentor | Mediterranean Basin, | IFA, molecular-based |
| | andersoni), mites, fleas, | United States | detection. |
| | lice | (southern California, | |
| | | Hawaii, and Texas) | |
| Powassan Virus | Hemaphysalis | Northeast and upper | Serological testing |
| | longicornis, Ixodes | midwest of the | |
| | scapularis, Ixodes cookei | United States and | |
| | | Russia | |
| Rickettsia rickettsii | Dermacentor variabilis, | North, Central, and | Indirect Immunoflorescence |
| | Dermacentor andersoni | South America | |
| Tick-borne | Tick bite (Ixodes ricinus, | Europe, Asia | Serological testing like ELISA, |
| Encephalitis Virus | Ixodes persulcatus) | | HI assay. |
| West Nile virus | Mosquito bite, Ixodes | Africa, Europe, the | ELISA, plaque reduction |
| | ricinus, O. moubata | Middle East, North | neutralization test (PRNT) |
| | | America and West | |
| | | Asia | |
| Coxsackie Virus | Amblyomma americanum | Worldwide | Viral culture, rtPCR (better |
| | | | sensitivity and specificity than |
| | | | viral culture) |
| Cytomegalovirus | Saliva, breast milk, blood | Endemic throughout | PCR, serology |
| | products | the world | |
| Epstein Barr virus | Bood transfusions | Worldwide | ELISA, IFA |
| Parvovirus | Tick (Haemaphysalis | Global | PCR |
| | longicornis) can retain the | | |
| | virus but cannot be | | |
| | infected in a laboratory | | |
| | condition, Respiratory | | |
| | tract, droplet infection, | | |
| | blood transfusion, person | | |
| | to person | | |
| Toxoplasma gondii | Tick (Dermacentor | Worldwide | ELISA, indirect fluorescent |
| | variabilis, Dermacentor | | antibody test, PCR. |
| | andersoni, Amblyomma | | |
| | americanum, | | |
| | Dermacentor reticulatus, | | |
| | Ixodes ricinus, Ixodes | | |
| | amblyomma, Amblyomma | | |
| | cajennense, Ornithodorus | | |
| | moubata and | | |
| | Haemaphysalis | | |
| | longicornis), blood | | |
| | transfusion, organ | | |
| | transplantation | | |
| HSV-1 | Oral secretions | Endemic throughout | Serological assays |
| | | the world | |
| HSV-2 | Skin contact | Endemic throughout | Serological assays |
| | | the world | |
| HHV-6 | Saliva, breast milk, blood | Worldwide | rtPCR |
| | products | | |

Table B provides a list of UniProt identifiers for exemplary tickborne pathogen proteins that can be used as probe molecules on the microarrays disclosed herein. The sequences associated with each UniProt identifier are the sequences in the UniProt database as of Sep. 8, 2023. In some embodiments, the probe molecule is a protein (e.g., a recombinant protein or peptide) selected from Table B.

TABLE B

| Antigen Name | Uniprot ID |
|---|---|
| Anaplasma Phagocytophilum Msp5 | A5H8H7 |
| Anaplasma Phagocytophilum p44 | D6RTX1 |
| Borreliella afzelii DbpA | Q9ZH82 |
| Borreliella afzelii OspA | Q0SLZ0 |
| Borreliella afzelii OspC | Q0SL36 |
| Borreliella burgdorferi DbpB | Q9R2P4 |
| Borreliella burgdorferi OspA | Q09086 |
| Borreliella burgdorferi OspC | O31120 |
| Borreliella burgdorferi p30 | P70831 |
| Borreliella burgdorferi p41 | Q57019 |
| Borreliella burgdorferi p66 | Q44881 |
| Borreliella garinii DbpA | Q9ZH95 |
| Borreliella garinii OspC | Q45175 |
| B. burgdorferi p100 | Q45018 |
| Parvovirus B19 VLP VP2 | Q76T42 |
| Parvovirus B19 VLP VP1/Vp2 Co Capsid | B3U154 |
| Borrelia mayonii nagB | A0AAC9KWS4 |
| Borrelia hermsii vsp13 | Q45207 |
| Borrelia coriaceae lon | W5SU95 |
| Babesia duncani coxIII | A0A385GNF5 |
| Bartonella elizabethae pth | J1A3B0 |
| Bartonella vinsonii virB3 | J1JYA6 |
| Borrelia maritima YjgP/YjgQ | A0A5J6WAW9 |
| Borrelia californiensis S1 | A0A7W9ZMQ9 |
| Borrelia bissettiae bptA | Q56NG9 |
| Borrelia valaisiana lipoprotein | C0R855 |
| Borrelia turcica enolase | A0A386PKF3 |
| Bartonella quintana hbpA | Q9KHA7 |
| Toxoplasma gondii MIC3 | A0A125YUG9 |
| Toxoplasma gondii P24 | P13403 |
| Toxoplasma gondii p30 | Q27298 |
| Powassan Virus polyprotein | Q04538 |
| West Nile Virus NS1 | A0A1S5XVW5 |
| Coxackievirus VP1 | Q9YLM7 |
| Tick-Borne Encephalitis Virus NS1 Protein | Q01299 |
| Parvovirus PepC (VP1 N-term.) | P07299 |
| Borrelia burgdorferi VlsE1 | Q5DVG3 |
| OSpB Controll | P17739 |
| Recombinant Rickettsia rickettsii (omp) | P16624 |
| Rickettsia typhi OmpB | P96989 |
| Borrelia miyamotoi GIpQ | A0A023PL72 |
| Chlamydia Pneumonia Recombinant | Q07430 |
| Streptolysin O | Q8P2T7 |
| HHV Strain 6B | P36320 |
| HHV Strain 6A | P36319 |
| HSV1 gG1 | P06484 |
| HSV2 gG2 | P13290 |
| Recombinant antigen pp150 for Cytomegalovirus | F5HIK0 |
| CMV Recombinant Glycoprotein B | P13201 |
| Recombinant Antigen EBNA1 | P03211 |
| Recombinant Antigen 23 | P25214 |
| Recombinant Cytomegalovirus pp65 protein | D3YS07 |

### Substrates

Also disclosed herein are substrates. In some embodiments, a substrate comprises a planar (e.g., 2-dimensional) layer. In some embodiments, the surface of a substrate comprises pillars for attachment or synthesis of molecules, e.g. peptides, or a first monomer building block. In other embodiments, a substrate includes a porous (i.e., a 3-dimensional) layer comprising functional groups for binding a first monomer building block. In some embodiments, a porous layer is added to the top of the pillars. In some embodiments, the substrate comprises a porous layer coupled to the planar layer. In other embodiments, the substrate comprises a plurality of pillars coupled to the planar layer.

In some embodiment, the planar layer can comprise any metal or plastic or silicon or silicon oxide or silicon nitride. In some embodiment, the planar layer has an upper surface and a lower surface. In some embodiments, the support layer is 1,000-2,000 angstroms thick. In some embodiments, the planar layer is about less than 500, 1,000, 2,000, 3,000, 4,000, 5,000, 6,000, 7,000, 8,000, 9,000, 10,000, 11,000, 12,000, or greater than 12,000 angstroms thick (or any integer in between). In some embodiments, the metal is chromium. In some embodiments, the metal is chromium, titanium, aluminum, tungsten, gold, silver, tin, lead, thallium, indium, or a combination thereof. In some embodiments, the planar layer is at least 98.5-99% metal. In some embodiments, the planar layer is 100% metal. In some embodiments, the planar layer is at least about greater than 90, 91, 92, 93, 94, 95, 96, 97, 98, 98.5, or 99% metal. In some embodiments, the planar layer is a homogenous layer of metal.

In some embodiments, a substrate surface is derivatized with free carboxylic acid groups. In other embodiments, a substrate surface is derivatized with free amine groups. In yet other embodiments, a substrate surface is derivatized with other free functional groups for solid state synthesis. A surface that is derivatized with free amine groups can be converted to free carboxylic acid groups by reacting the amine with one carboxylic acid group of a molecule having at least two free carboxylic acid groups. For example, by using carbodiimide one carboxylic acid group is first activated to form an intermediate O-acylisourea that then further reacts with the free amine groups for an amide bond and attached to the substrate surface. In some embodiments, the molecule with multiple carboxylic acid groups includes, but is not limited to, succinic anhydride, polyethylene glycol diacid, benzene-1,3,5-tricarboxylic acid, benzenehexacarboxylic acid and carboxymethyl dextran. For example, the free carboxylic acid or free amine groups bind amino acids, peptides or proteins during peptide synthesis and protein coupling. In another example, the free functional groups bind to linker molecules that couple (“link”) other probe molecules or biomarkers to the substrate. In some embodiments, a coupling molecule is attached to the surface of at least one pillar. In other embodiments, a coupling molecule is attached to the surface of each pillar.

In some embodiments, a polymer is in contact with the surface of at least one of said pillars. In other embodiments, a polymer is in contact with the surface of each pillar. In some embodiments, a gelatinous form of a polymer is in contact with the surface of at least one of said pillars. In some embodiments, a solid form of a water soluble polymer is in contact with the surface of at least one of said pillars.

In some embodiments, the substrate surface comprises silicon dioxide for contacting the surface with a photoactive coupling formulation comprising a photoactive compound, a coupling molecule, a coupling reagent, a polymer, and a solvent, wherein the contracting is followed by applying ultraviolet light to positionally-defined locations located on the top of the surface and in contact with the photoactive coupling formulation.

In some embodiments, the substrate surface is a material or group of materials having rigidity or semi-rigidity. In some embodiments, the substrate surface can be substantially flat, although in some embodiments it can be desirable to physically separate synthesis regions for different molecules or features with, for example, wells, raised regions, pins, pillars, etched trenches, or the like. In certain embodiments, the substrate surface may be porous. Surface materials can include, for example, silicon, bio-compatible polymers such as, for example poly(methyl-methacrylate) (PMMA) and polydimethylsiloxane (PDMS), glass, SiO₂ (such as a thermal oxide silicon wafer used by the semiconductor industry), quartz, silicon nitride, functionalized glass, gold, platinum, and aluminum.

Derivatized substrate surfaces include, for example, amino-derivatized glass, carboxy-derivatized glass, and hydroxyl-derivatized glass. Additionally, a surface may optionally be coated with one or more layers to provide a second surface for molecular attachment or derivatization, increased or decreased reactivity, binding detection, or other specialized application. Substrate surface materials and/or layer(s) can be porous or non-porous. For example, a substrate surface comprises porous silicon.

### Pillar Substrate

In some embodiments, a substrate comprises a planar layer comprising a metal and having an upper surface and a lower surface; and a plurality of pillars operatively coupled to the planar layer in positionally-defined locations, wherein each pillar has a planar surface extended from the planar layer, wherein the distance between the surface of each pillar and the upper surface of the planar layer is between about 1,000-5,000 angstroms, and wherein the plurality of pillars are present at a density of greater than about 10,000/cm². In other embodiments, the distance between the surface of each pillar and the upper surface of the planar layer can be between about less than 1,000, 2,000, 3,000, 3,500, 4,500, 5,000, or greater than 5,000 angstroms (or any integer in between).

In some embodiments, the surface of each pillar is parallel to the upper surface of the planar layer. In some embodiments, the surface of each pillar is substantially parallel to the upper surface of the planar layer.

In some embodiments, the distance between the surface of each pillar and the lower surface of the planar layer is 2,000-7,000 angstroms. In other embodiments, the distance between the surface of each pillar and the lower surface of the planar layer is about less than 500, 1,000, 2,000, 3,000, 4,000, 5,000, 6,000, 7,000, 8,000, 9,000, 10,000, 11,000, 12,000, or greater than 12,000 angstroms (or any integer in between). In yet other embodiments, the distance between the surface of each pillar and the lower surface of the planar layer is 7,000, 3,000, 4,000, 5,000, 6,000, or 7,000 angstroms (or any integer in between).

In some embodiments, the plurality of pillars are present at a density of greater than 10, 20, 30, 40, 50, 60, 70, 80, 90, 100, 200, 300, 400, 500, 1,000, 2,000, 3,000, 4,000, 5,000, 6,000, 7,000, 8,000, 9,000, 10,000, 11,000, or 12,000/cm² (or any integer in between). In other embodiments, the plurality of pillars are present at a density of greater than 10,000/cm². In yet other embodiments, the plurality of pillars are present at a density of about 10,000/cm² to about 2.5 million/cm² (or any integer in between). In some embodiments, the plurality of pillars are present at a density of greater than 2.5 million/cm².

In some embodiments, the surface area of each pillar surface is at least 1 μm². In other embodiments, the surface area of each pillar surface can be at least 0.1, 0.5, 12, 3, 4, 5, 6, 7, 8, 9, 10, 15, 20, 25, 30, 35, 40, 45, or 50 μm² (or any integer in between). In yet other embodiments, the surface area of each pillar surface has a total area of less than 10,000 μm². In yet other embodiments, the surface area of each pillar surface has a total area of less than 500, 1,000, 2,000, 3,000, 4,000, 5,000, 6,000, 7,000, 8,000, 9,000, 10,000, 11,000, or 12,000 μm² (or any integer in between). In some embodiments, the surface of each pillar is square or rectangular in shape.

In some embodiments, the center of each pillar is at least 2,000 angstroms from the center of any other pillar. In other embodiments, the center of each pillar is at least about 500, 1,000, 2,000, 3,000, or 4,000 angstroms (or any integer in between) from the center of any other pillar. In yet other embodiments, the center of each pillar is at least about 2 μm to 200 μm from the center of any other pillar.

In some embodiments, at least one or each pillar comprises silicon. In other embodiments, at least one or each pillar comprises silicon dioxide or silicon nitride. In some of these embodiments, at least one or each pillar is at least 90, 91, 92, 93, 94, 95, 96, 97, 98, 98.5, or 99% silicon dioxide.

In some embodiments, the metal of the planar layer is chromium. In other embodiments, the metal is chromium, titanium, aluminum, tungsten, gold, silver, tin, lead, thallium, indium, or a combination thereof. In some embodiments, the planar layer is at least 98.5-99% (by weight) metal. In other embodiments, the planar layer is 100% metal. In yet other embodiments, the planar layer is at least about greater than 90, 91, 92, 93, 94, 95, 96, 97, 98, 98.5, or 99% metal. In some embodiments, the planar layer is a homogenous layer of metal.

In some embodiments, the surface of at least one of said pillars of the substrate is derivatized. In some embodiments, a substrate can include a polymer chain attached to the surface of at least one of said pillars. In some embodiments, the polymer chain comprises a peptide chain. In some embodiments, the attachment to the surface of said at least one pillar is via a covalent bond.

In some embodiments, the substrate can be coupled to a silicon dioxide layer. The silicon dioxide layer can be about 0.5 μm to 3 μm thick. In some embodiments, the substrate can be coupled to a wafer, e.g., a silicon wafer. The silicon wafer can be about 700 μm to 750 μm thick.

### Porous Layers Substrate

In another embodiments, a substrate comprises a porous layer coupled to a plurality of pillars, wherein the porous layer comprises functional groups for attachment of a molecule to the substrate, and wherein the plurality of pillars are coupled to a planar layer in positionally-defined locations, each pillar having a planar surface extended from the planar layer by the distance between the surface of each pillar and the upper surface of the planar layer that is between about 1,000-5,000 angstroms, and the plurality of pillars are present at a density of greater than about 10,000/cm².

Porous layers that can be used are flat, permeable, polymeric materials of porous structure that have a carboxylic acid functional group (that is native to the constituent polymer or that is introduced to the porous layer) for attachment of the first peptide building block. For example, a porous layer can be comprised of porous silicon with functional groups for attachment of a polymer building block attached to the surface of the porous silicon. In another example, a porous layer can comprise a cross-linked polymeric material. In some embodiments, the porous layer can employ polystyrenes, saccharose, dextrans, polyacryloylmorpholine, polyacrylates, polymethylacrylates, polyacrylamides, polyacrylolpyrrolidone, polyvinylacetates, polyethyleneglycol, agaroses, sepharose, other conventional chromatography type materials and derivatives and mixtures thereof. In some embodiments, the porous layer building material is selected from: poly(vinyl alcohol), dextran, sodium alginate, poly(aspartic acid), poly(ethylene glycol), poly(ethylene oxide), poly(vinyl pyrrolidone), poly(acrylic acid), poly(acrylic acid)-sodium salt, poly(acrylamide), poly(N-isopropyl acrylamide), poly(hydroxyethyl acrylate), poly(acrylic acid), poly(sodium styrene sulfonate), poly(2-acrylamido-2-methyl-1-propanesulfonic acid), polysaccharides, and cellulose derivatives. Preferably the porous layer has a porosity of 10-80%. In one embodiment, the thickness of the porous layer ranges from 0.01 μm to about 1,000 μm. Pore sizes included in the porous layer may range from 2 nm to about 100 μm.

In another embodiment the porous layer comprises a porous polymeric material having a porosity from 10-80%, wherein reactive groups are chemically bound to the pore surfaces and are adapted in use to interact, e.g. by binding chemically, with a reactive species, e.g., deprotected monomeric building blocks or polymeric chains. In one embodiment the reactive group is a free carboxylic acid or a free amine group. For example, the carboxylic acid group is free to bind an unprotected amine group of an amino acid, peptide or polypeptide for peptide synthesis.

### Linker Molecules

In some embodiments, the substrate surface is coupled to a plurality of linker molecules. A linker molecule is a molecule inserted between a substrate surface disclosed herein and a first coupling molecule that is e.g. the N-terminal amino acid of a peptide being synthesized. A linker molecule does not necessarily convey functionality to the resulting peptide, such as molecular recognition functionality, but can instead elongate the distance between the surface and the synthesized peptide to enhance the exposure of the peptide's functionality region(s) on the surface.

In some embodiments, a linker can be about 4 to about 40 atoms long to provide exposure. The linker molecules can be, for example, aryl acetylene, ethylene glycol oligomers containing 2-10 monomer units, diamines, diacids, amino acids, and combinations thereof. Examples of diamines include ethylene diamine and diamino propane.

Alternatively, linkers can be the same molecule type as that being synthesized (e.g., nascent polymers or various coupling molecules), such as polypeptides and polymers of amino acid derivatives such as for example, amino hexanoic acids. In some embodiments, a linker molecule is a molecule having a carboxylic group at a first end of the molecule and a protecting group at a second end of the molecule. In some embodiments, the protecting group is a boc or Fmoc protecting group. In some embodiments, a linker molecule comprises an aryl-acetylene, a polyethyleneglycol (PEGs), a nascent polypeptide, a diamine, a diacid, a peptide, or combinations thereof.

The unbound portion of a linker molecule, or free end of the linker molecule, can have a reactive functional group which is blocked, protected, or otherwise made unavailable for reaction by a removable protective group, e.g., boc or Fmoc as noted above. The protecting group can be bound to a monomer, a polymer, or a linker molecule to protect a reactive functionality on the monomer, polymer, or linker molecule. Protective groups that can be used include all acid and base labile protecting groups. For example, peptide amine groups can be protected by tert-butyloxycarbonyl (boc) or benzyloxycarbonyl (CBZ), both of which are acid labile, or by 9-fluorenylmethoxycarbonyl (Fmoc), which is base labile.

Additional protecting groups that can be used include acid labile groups for protecting amino moieties: tert-amyloxycarbonyl, adamantyloxycarbonyl, 1-methylcyclobutyloxycarbonyl, 2-(p-biphenyl) propyl(2)oxycarbonyl, 2-(p-phenylazophenylyl) propyl(2)oxycarbonyl, alpha,alpha-dimethyl-3,5-dimethyloxybenzyloxy-carbonyl, 2-phenylpropyl(2)oxycarbonyl, 4-methyloxybenzyloxycarbonyl, furfuryloxycarbonyl, triphenylmethyl (trityl), p-toluenesulfenylaminocarbonyl, dimethylphosphinothioyl, diphenylphosphinothioyl, 2-benzoyl-1-methylvinyl, o-nitrophenylsulfenyl, and 1-naphthylidene; as base labile groups for protecting amino moieties: 9 fluorenylmethyloxycarbonyl, methylsulfonylethyloxycarbonyl, and 5-benzisoazolylmethyleneoxycarbonyl; as groups for protecting amino moieties that are labile when reduced: dithiasuccinoyl, p-toluene sulfonyl, and piperidino-oxycarbonyl; as groups for protecting amino moieties that are labile when oxidized: (ethylthio) carbonyl; as groups for protecting amino moieties that are labile to miscellaneous reagents, the appropriate agent is listed in parenthesis after the group: phthaloyl (hydrazine), trifluoroacetyl (piperidine), and chloroacetyl(2-aminothiophenol); acid labile groups for protecting carboxylic acids: tert-butyl ester; acid labile groups for protecting hydroxyl groups: dimethyltrityl. (See also, Greene, T. W., Protective Groups in Organic Synthesis, Wiley-Interscience, NY, (1981)).

In some embodiments, the linker molecule is silane-(boc), where (boc) represents a tert-butyloxycarbonyl protecting group. In some embodiments, the linker molecule is silane-Gly-PEG(boc). In some embodiments, the linker molecule is silane-Gly-PEG-PEG(boc). In some embodiments, the linker molecule is silane-Gly-(PEG(boc)) 2. In some embodiments, the linker molecule is silane-PEG-Gly(boc). In some embodiments, the linker molecule is silane-Gly-cyc-PEG(boc), where Gly-cyc represents a glycine chain with a cyclic glycine chain conformation. In some embodiments, the linker molecule is silane-Gly-(PEG(boc)) 4.

In some embodiments, linker molecules attached to the surface of each pillar of the pillar substrate described above comprise a free amine or free carboxylic acid group. In other embodiments, linker molecules attached to the surface of at least one pillar of the pillar substrate comprise a free amine or free carboxylic acid group. In some embodiments, a linker molecule having a protecting group is attached to the surface of each pillar. In other embodiments, a linker molecule having a protecting group is attached to the surface of at least one pillar.

### Linker Formulations

Also disclosed herein is a linker formulation used for reacting a linker molecule with the substrate. A linker formulation can include components such as a linker molecule, a polymer, a solvent and a coupling reagent.

In some embodiments, a linker molecule is about 0.5-5 weight % of the total formulation concentration. In some embodiments, a linker molecule is about less than 0.1, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, or greater than 5.0 weight % of the total formulation concentration.

In some embodiments, the polymer is 1 weight % polyvinyl alcohol and 2.5 weight % poly vinyl pyrrollidone, the linker molecule is 1.25 weight % polyethylene oxide, the coupling reagent is 1 weight % 1-ethyl-3-(3-dimethylaminopropyl) carbodiimide, and the solvent includes water. In some embodiments, the polymer is 0.5-5 weight % polyvinyl alcohol and 0.5-5 weight % poly vinyl pyrrollidone, the linker molecule is 0.5-5 weight % polyethylene oxide, the coupling reagent is 0.5-5 weight % 1-ethyl-3-(3-dimethylamino-propyl) carbodiimide, and the solvent includes water.

In some embodiments, the polymer is a polyvinyl pyrrolidone and/or a polyvinyl alcohol. The general structure of polyvinyl alcohol is as follows, where n is any positive integer greater than 1:

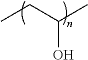

In some embodiments, the polymer is about 0.5-5 weight % of the total formulation concentration. In some embodiments, a water soluble polymer is about less than 0.1, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, or greater than 5.0 weight % of the total formulation concentration.

In some embodiments, the solvent is water, an organic solvent, or a combination thereof. In some embodiments, the organic solvent is N-methyl pyrrolidone, dimethyl formamide, dichloromethane, dimethyl sulfoxide, or a combination thereof. In some embodiments, the solvent is about 80-90 weight % of the total formulation concentration. In some embodiments, the solvent is about less than 70, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or greater than 99 weight % of the total formulation concentration.

In some embodiments, the coupling reagent is carbodiimide. In some embodiments, a coupling reagent is a water soluble triazole. In some embodiments, a coupling reagent is 1-ethyl-3-(3-dimethylaminopropyl) carbodiimide. In some embodiments, the coupling reagent is about 0.5-5 weight % of the total formulation concentration. In some embodiments, the coupling reagent is about less than 0.1, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, or greater than 5.0 weight % of the total formulation concentration.

### Methods

### Method of Manufacturing Substrates

Also disclosed herein are methods for making substrates for the microarray or array. In some embodiments, a method of producing a substrate can include coupling a porous layer to a support layer. The support layer can comprise any metal or plastic or silicon or silicon oxide or silicon nitride. In one embodiment, the substrate comprises multiple carboxylic acid substrates attached to the substrate for binding peptides during peptide synthesis and protein coupling. In some embodiments, a method of producing a substrate can include coupling a porous layer to a plurality of substrate pillars, wherein the porous layer comprises functional groups for attachment of a compound to the substrate, wherein the plurality of substrate pillars are coupled to a planar layer in positionally-defined locations, wherein each substrate pillar has a planar surface extended from the planar layer, wherein the distance between the surface of each substrate pillar and the upper surface of the planar layer is between about 1,000-5,000 angstroms, and wherein the plurality of substrate pillars are present at a density of greater than about 10,000/cm².

In some embodiments, the surface of each substrate pillar is parallel to the upper surface of the planar layer. In some embodiments, the surface of each substrate pillar is substantially parallel to the upper surface of the planar layer.

In some embodiments, a method of preparing a substrate surface can include obtaining a surface comprising silicon dioxide and contacting the surface with a photoactive coupling formulation comprising a photoactive compound, a coupling molecule, a coupling reagent, a polymer, and a solvent; and applying ultraviolet light to positionally-defined locations located on the top of the surface and in contact with the photoactive formulation.

### Methods of Manufacturing Microarrays

Also disclosed herein are methods for manufacturing microarrays. In some embodiments, the microarrays disclosed herein can be synthesized in situ on a surface, e.g., the substrate disclosed herein. In some instances, the microarrays are made using photolithography. For example, the substrate is contacted with a photoactive coupling solution. Masks can be used to control radiation or light exposure to specific locations on a surface provided with free linker molecules or free coupling molecules having protecting groups. In the exposed locations, the protecting groups are removed, resulting in one or more newly exposed reactive moieties on the coupling molecule or linker molecule. The desired linker or coupling molecule is then coupled to the unprotected attached molecules, e.g., at the carboxylic acid group. The process can be repeated to synthesize a large number of features in specific or positionally-defined locations on a surface (see, for example, U.S. Pat. No. 5,143,854 to Pirrung et al., U.S. Patent Application Publication Nos. 2007/0154946 (filed on Dec. 29, 2005), 2007/0122841 (filed on Nov. 30, 2005), 2007/0122842 (filed on Mar. 30, 2006), 2008/0108149 (filed on Oct. 23, 2006), and 2010/0093554 (filed on Jun. 2, 2008), each of which is herein incorporated by reference).

In some embodiments, features at a positionally-defined location on a microarray are synthesized in situ on the microarray surface. In some embodiments, features at a positionally-defined location on a microarray comprise microchips comprising probe molecules attached to the microchip surface, wherein the microchip is placed on or attached to a second surface (e.g., a support surface such as pillar plate surface) at specific locations.

In some embodiments, a method of producing a three-dimensional microarray of features, can include obtaining a porous layer attached to a surface; and attaching the features to the porous layer, said features each comprising a collection of peptide chains of determinable sequence and intended length, wherein within an individual feature, the fraction of peptide chains within said collection having the intended length is characterized by an average coupling efficiency for each coupling step of at least about 98%. In some embodiments, the features are attached to the surface using a photoactive coupling formulation, comprising a photoactive compound, a coupling molecule, a coupling reagent, a polymer, and a solvent. In some embodiments, the features are attached to the surface using a photoactive coupling formulation disclosed herein. In some embodiments, the photoactive coupling formulation is stripped away using water.

In one embodiment, described herein is a process of manufacturing an microarray. A surface comprising attached carboxylic acid groups is provided. The surface is contacted with a photoactive coupling solution comprising a photoactive compound, a coupling molecule, a coupling reagent, a polymer, and a solvent. The surface is exposed to ultraviolet light in a deep ultra violet scanner tool according to a pattern defined by a photomask, wherein the locations exposed to ultraviolet light undergo photo base generation due to the presence of a photobase generator in the photoactive coupling solution. The expose energy can be from 1 mJ/cm² to 100 mJ/cm² in order to produce enough photobase.

The surface is post baked upon exposure in a post exposure bake module. Post exposure bake acts as a chemical amplification step. The baking step amplifies the initially generated photobase and also enhances the rate of diffusion to the substrate. The post bake temperature can vary between 75° Celsius to 115° Celsius, depending on the thickness of the porous surface, for at least 60 seconds and not usually exceeding 120 seconds. The free carboxylic acid group is coupled to the deprotected amine group of a free peptide or polypeptide, resulting in coupling of the free peptide or polypeptide to the carboxylic acid group attached to the surface. This surface may be a porous surface. The synthesis of peptides coupled to a carboxylic acid group attached to the surface occurs in an N→C synthesis orientation, with the amine group of free peptides attaching to carboxylic acid groups bound to the surface of the substrate. Alternatively, a diamine linker may be attached to a free carboxylic acid group to orient synthesis in a C→N direction, with the carboxylic acid group of free peptides attaching to amine groups bound to the surface of the substrate.

The photoactive coupling solution can now be stripped away. In some embodiments, provided herein is a method of stripping the photoresist completely with deionized (DI) water. This process is accomplished in a developer module. The wafer is spun on a vacuum chuck for, e.g., 60 seconds to 90 seconds and deionized water is dispensed through a nozzle for about 30 seconds.

The photoactive coupling formulation may be applied to the surface in a coupling spin module. A coupling spin module can typically have 20 nozzles or more to feed the photoactive coupling formulation. These nozzles can be made to dispense the photoactive coupling formulation by means of pressurizing the cylinders that hold these solutions or by a pump that dispenses the required amount. In some embodiments, the pump is employed to dispense 5-8 cc of the photoactive coupling formulation onto the substrate. The substrate is spun on a vacuum chuck for 15-30 seconds and the photoactive coupling formulation is dispensed. The spin speed can be set to 2000 rpm to 2500 rpm.

Optionally, a cap film solution coat is applied on the surface to prevent the non-reacted amino groups on the substrate from reacting with the next coupling molecule. The cap film coat solution can be prepared as follows: a solvent, a polymer, and a coupling molecule. The solvent that can be used can be an organic solvent like N-methyl pyrrolidone, dimethyl formamide, or combinations thereof. The capping molecule is typically acetic anhydride and the polymer can be polyvinyl pyrrolidone, polyvinyl alcohol, polymethyl methacrylate, poly-(methyl-isopropenyl)-ketone, or poly-(2-methyl-pentene-1-sulfone). In some embodiments, the capping molecule is ethanolamine.

This process is done in a capping spin module. A capping spin module can include one nozzle that can be made to dispense the cap film coat solution onto the substrate. This solution can be dispensed through pressurizing the cylinder that stores the cap film coat solution or through a pump that precisely dispenses the required amount. In some embodiments, a pump is used to dispense around 5-8 cc of the cap coat solution onto the substrate. The substrate is spun on a vacuum chuck for 15-30 seconds and the coupling formulation is dispensed. The spin speed can be set to 2000 to 2500 rpm.

The substrates with the capping solution are baked in a cap bake module. A capping bake module is a hot plate set up specifically to receive wafers just after the capping film coat is applied. In some embodiments, provided herein is a method of baking the spin coated capping coat solution in a hot plate to accelerate the capping reaction significantly. Hot plate baking generally reduces the capping time for amino acids to less than two minutes.

The byproducts of the capping reaction are stripped in a stripper module. A stripper module can include several nozzles, typically up to 10, set up to dispense organic solvents such as acetone, isopropyl alcohol, N-methyl pyrrolidone, dimethyl formamide, DI water, etc. In some embodiments, the nozzles can be designated for acetone followed by isopropyl alcohol to be dispensed onto the spinning wafer. The spin speed is set to be 2000 to 2500 rpm for around 20 seconds.

This entire cycle can be repeated as desired with different coupling molecules each time to obtain a desired sequence.

In some embodiments, a microarray comprising a surface of free carboxylic acids is used to synthesize polypeptides in an N→C orientation. In one embodiment, the carboxylic acids on the surface of the substrate are activated (e.g., converted to a carbonyl) to allow them to bind to free amine groups on an amino acid. In one embodiment, activation of carboxylic acids on the group of the surface can be done by addition of a solution comprising a carbodiimide or succinimide to the surface of the microarray. In some embodiments, carboxylic acids can be activated by addition of a solution comprising 1-ethyl-3-(3-dimethyl-aminopropyl)-carbodiimide (EDC), N-hydroxysuccinimide (NHS), 1,3-diisopropyl-carbodiimide (DIC), hydroxybenzotriazole (HOBt), O-(7-azabenzotriazol-1-yl)-N,N,N′,N′-tetramethyluronium hexafluorophosphate (HATU), benzotriazol-1-yl-oxytripyrrolidinophosphonium hexafluorophosphate (PyBOP), or N,N-diisopropylethylamine (DIEA) to the surface of the microarray. The activation solution is washed away and the surface of the microarray is prepared for addition of an amino acid layer (i.e., one amino acid at each activated carboxylic acid group). Carboxylic acid groups remain activated for up to 2, 3, 4, 5, 6, 7, 8, 9, or 10 hours.

Addition of a solution comprising an amino acid with a free amine group to the activated carboxylic acid surface of the microarray results in binding of a single amino acid to each carboxylic acid group. In some embodiments, the amino acid comprises an amino acid with protected amine groups. Using a photosensitive chemical reaction, the protecting group can be removed from the amine group of selected amino acids at site-specific locations using a reticle. For example, Fmoc-protected amino acids are mixed in a solution comprising a photobase. Upon exposure of the solution on the microarray to a specific frequency of light at site-specific locations, the photobase will release a base which will deprotect the amino acid, resulting in coupling of the amino acid to the activated carboxylic acid group on the surface of the microarray. Another method of generating a base is through the use of a photoacid generator. In some embodiments, the photoacid generator is N-boc-piperidine or 1-boc-4-piperazine.

After a completed layer of amino acids is coupled, remaining uncoupled activated carboxylic acids are capped to prevent nonspecific binding of amino acids on subsequent synthesis steps. The steps of activation, addition of an amino acid layer, and capping are repeated as necessary to synthesize the desired polypeptides at specific locations on the microarray.

In one embodiment, peptides synthesized in the N→C terminus direction can be capped with a diamine molecule to enhance binding properties of selected polypeptide sequences to a biological molecule, e.g., an antibody. In other embodiments, peptides synthesized in the C→N direction can be capped with a dicarboxylic acid molecule to enhance binding properties of selected sequences to a biological molecule.

While synthesizing polypeptides in parallel on the surface of a microarray, the method described herein ensures complete activation of carboxylic acid on the surface of the microarray. Due to stability of the activated ester for an extended period of time, 2, 3, 4, 5, 6, 7, 8, 9, 10, 11, 12, 13, 14, 15, 16, 17, 18, 19, 20, 21, 22, 23, 24, 25 or more coupling cycles may be completed after a single activation step (e.g., to couple an entire layer of 2-25 or more different amino acids at different locations on the microarray). As the coupling occurs during hard bake (heating in a hot plate at 85-90° Celsius for 90 seconds immediately after coating) and due to the presence of excess amino acid in the solution, complete 100% deprotection of Fmoc-protected amino acid may not be required for significantly high coupling yields. After addition of all amino acids and capping, all free activated carboxylic acids are either coupled or capped, thus resulting in high efficiency and accuracy of polypeptide synthesis.

### Methods of Use of Microarrays

Also disclosed herein are methods of using substrates, formulations, and/or arrays and microarrays. Uses of the arrays and microarrays disclosed herein can include research applications, therapeutic purposes, medical diagnostics, and/or stratifying one or more patients.

In one aspect, provided herein are methods of identifying a tickborne disease or pathogen in a subject, comprising: contacting a sample from the subject with the microarray disclosed herein; and analyzing binding of antibodies in the sample to the features on the microarray to determine whether the subject has the tickborne disease or pathogen.

In some embodiments, the tickborne disease or pathogen is Lyme disease, Borrelia burgdorferi, Borrelia mayonii, Borrelia afzelii, Borrelia garinii, Borrelia Bavariensis, Borrelia speielmanii, Borrelia hemsii, Borrelia Turicatae, Borrelia miyamotoi, Borrelia andersonii, Borrelia maritima, Borrelia californiensis, Borrelia bissettiae, Borrelia lusitaniae, Borrelia valaisiana, Borrelia yangtzensis, Borrelia turcia, Babsia microti, Bartonella henselae, Anaplasma phagocytophilum, Ehrlichia chaffeensis, Rickettsia typhi, Rickettsia rickettsii, Powassan virus, Tick-borne encephalitis virus, West Nile virus, Coxsackie virus, Cytomegalovirus, Epstein Barr virus, Parvovirus B19, Toxoplasma, gondii, HSV-1, or HHV-6.

In some embodiments, the method comprises a sensitivity of detection of IgM antibodies that bind to the tickborne disease or pathogen with a sensitivity of at least 25%, 55%, 60%, 65%, 70%, 75%, 80%, 85%, 90%, 91%, 92%, 93%, 94%, 95%, 96%, 97%, 98%, or 99%. In some embodiments, the method comprises a specificity of detection of IgM antibodies that bind to the tickborne disease or pathogen with a sensitivity of at least 95%, 96%, 97%, 98%, 99%, or 100%. In some embodiments, the method comprises a sensitivity of detection of IgG antibodies that bind to the tickborne disease or pathogen with a sensitivity of at least 80%, 83%, 85%, 87%, 90%, 91%, 92%, 93%, 94%, 95%, 96%, 97%, 98%, 99%, or 100%. In some embodiments, the method comprises a specificity of detection of IgM antibodies that bind to the tickborne disease or pathogen with a sensitivity of at least 95%, 96%, 97%, 98%, 99%, or 100%. In some embodiments, the method comprises a sensitivity of detection of IgG and IgM antibodies that bind to the tickborne disease or pathogen with a sensitivity of at least 85%, 87%, 90%, 91%, 92%, 93%, 94%, 95%, 96%, 97%, 98%, 99%, or 100%. In some embodiments, the method comprises a specificity of detection of IgG and IgM antibodies that bind to the tickborne disease or pathogen with a sensitivity of at least 95%, 96%, 97%, 98%, 99%, or 100%.

In some embodiments, the sample is blood or serum. In some embodiments, the subject is human. In some embodiments, the method comprises analysis of binding of IgG, IgM or IgG and IgM antibodies present in the sample to the features on the microarray.

In another aspect, provided herein are methods for obtaining feature binding data, comprising obtaining the microarray disclosed herein, contacting the microarray with a sample comprising a plurality of ligands for at least a subset of the features under conditions that promote ligand binding; and imaging the microarray to identify binding of the plurality of ligands to the features of the microarray.

In some embodiments, the sample is from a subject, optionally wherein the sample is blood or serum, optionally wherein the subject is human. In some embodiments, the ligands are IgG, IgM, or IgG and IgM antibodies.

In some embodiments, a total number of features is at least about 100, 200, 300, 400, 10,000, 50,000, 100,000, 500,000, at least about 1,000,000, at least about 2,000,000, or at least about 18,000,000.

In some embodiments, the microarray has an area that is less than or equal to 0.2 square millimeters, less than or equal to 1 square millimeters, less than or equal to 10 square millimeters, less than or equal to 100 square millimeters, or less than or equal to 150 square millimeters.

In some embodiments, the sample has a volume that is less than or equal to 100 μL, less than or equal to 50 μL, less than or equal to 25 μL, less than or equal to 10 μL, less than or equal to 5 μL, less than or equal to 1.5 μL, or less than or equal to 1 μL.

In some embodiments, an elapsed time from sample contacting to finishing the imaging is equal or less than 20 minutes, equal or less than 5 minutes, equal or less than 1 minute, equal or less than 2-seconds, equal or less than 10 seconds, equal or less than 1 second. In some embodiments, a coefficient of variation of data obtained from the array is not greater than 5 percent, not greater than 2 percent, not greater than 1 percent.

In some embodiments, the microarray comprises at least 1,000,000 features per square centimeter, at least 10,000,000 features per square centimeter, or at least 15,000,000 features per square centimeter.

In some embodiments, the contacting occurs at a concentration of the plurality of ligands that is less or equal than 1,000 μg/ml in the sample, less or equal than 10 μg/ml in the sample, less or equal than 1 μg/ml in the sample, less or equal than 0.1 μg/ml in the sample, less or equal than 10 ng/ml in the sample, less or equal than 1 ng/ml in the sample, less or equal than 5 μg/ml in the sample. In some embodiments, the contacting occurs at a concentration of the plurality of ligands that is within the range of approximately 1 μg/ml to approximately 1,000 μg/ml in the sample. In some embodiments, the imaging comprises identifying binding of at least 1,000 ligands, at least 100,000 ligands, at least 1,000,000 ligands, at least 10,000,000 ligands, at least 15,000,000 ligands, or at least 100,000,000 ligands to the features of the microarray.

In some embodiments, the ligands comprise an antibody or antigen-binding fragment. In some embodiments, the antibody or antigen-binding fragment is an IgG antibody or IgM antibody or a combination thereof.

Any of the arrays and microarrays described herein can be used as a research tool or in a research application. In one embodiment, arrays and microarrays can be used for high throughput screening assays. For example, antigenic substrates (i.e., peptides on a peptide array and/or microarray described herein or lysates or sonicates from whole cell on an array and/or microarray) can be tested by subjecting the microarray to sample and identifying the binding of a protein of interest to the antigenic substrate(s) on the microarray, e.g., by detecting at least one change among the features of the microarray.

Microarrays can also be used in screening assays for ligand binding, to determine substrate specificity, or for the identification of peptides that inhibit or activate proteins. Labeling techniques, protease assays, as well as binding assays useful for carrying out these methodologies are generally well-known to one of skill in the art.

In some embodiments, an array or microarray can be used to represent a known protein sequence as a sequence of overlapping peptides. For example, the amino acid sequence of a known protein is divided into overlapping sequence segments of any length and of any suitable overlapping frame, and peptides corresponding to the respective sequence segments are in-situ synthesized as disclosed herein. The individual peptide segments so synthesized can be arranged starting from the amino terminus of the known protein.

In another example, a microarray can be used to identify one or more biomarkers. Biomarkers can be used for the diagnosis, prognosis, treatment, and management of diseases. Biomarkers may be expressed, or absent, or at a different level in an individual, depending on the disease condition, stage of the disease, and response to disease treatment. Biomarkers can be, e.g., DNA, RNA, proteins (e.g., enzymes such as kinases), sugars, salts, fats, lipids, or ions.

In another embodiment, a microarray can be used to identify drug candidates for therapeutic use. For example, when one or more epitopes for specific antibodies are determined by an assay (e.g., a binding assay such as an ELISA), the epitopes can be used to develop a drug (e.g., a monoclonal neutralizing antibody) to target antibodies in disease.

In one embodiment, also provided are microarrays for use in medical diagnostics. An array can be used to determine a response to administration of drugs or vaccines. For example, an individual's response to a vaccine can be determined by detecting the antibody level of the individual by using a microarray with peptides representing epitopes recognized by the antibodies produced by the induced immune response. Another diagnostic use is to test an individual for the presence of biomarkers, wherein samples are taken from a subject and the sample is tested for the presence of one or more biomarkers.

In some embodiments, a method of detecting the presence or absence of a molecule of interest (e.g., a protein, an antibody, or any other ligand) in a sample can include obtaining a microarray disclosed herein and contacted with a sample suspected of comprising the molecule of interest; and determining whether the molecule of interest is present in the sample by detecting the presence or absence of binding to one or more features of the microarray.

In some embodiments, a molecule of interest (e.g., an antibody) can be detected within a sample that has a volume that is less than or equal to 100, 50, 10, 5, 1.5, or 1 μL. In some embodiments, the elapsed time from the sample contacting to detection of a molecule of interest is less than 40, 30, 20, 19, 18, 17, 16, 15, 14, 13, 12, 11, 10, 9, 8, 7, 6, 5, 4, 3, 2, or 1 minutes. In some embodiment, a molecule of interest can be detected at a concentration in the contacted sample that falls within the range of about 1 μg/ml to 1,000 μg/ml.

In some embodiments, the protein of interest may be obtained from a bodily fluid, such as amniotic fluid, aqueous humor, vitreous humor, bile, blood, blood serum, breast milk, cerebrospinal fluid, cerumen, chyle, endolymph, perilymph, feces, female ejaculate, gastric acid, gastric juice, lymph, mucus, peritoneal fluid, pleural fluid, pus, saliva, sebum, semen, sweat, synovial fluid, tears, vaginal secretion, vomit, or urine.

In some embodiments, a method of identifying a vaccine candidate can include obtaining a microarray disclosed herein contacted with a sample derived from a subject previously administered the vaccine candidate, wherein the sample comprises a plurality of antibodies; and determining the binding specificity of the plurality of antibodies to one or more features of the microarray. In some embodiments, the features comprise a plurality of distinct, nested, overlapping peptide chains comprising subsequences derived from a source protein having a known sequence.

### Remote Microarray Analysis

In some embodiments, a diagnostic device comprising a chip array is located in a third party location (e.g., a reference lab or a diagnostic lab). In some embodiments, the assay is performed in one or several of the third party locations and the patient samples are barcoded. The raw data output from the chip array is input into an analyzer at a user-controlled location. This transfer may be through a VPN or via any other remote data transfer method. In some embodiments, the raw data is stored in a temporary user-controlled database for a finite time. A test report is generated and the results are provided to the third party. Any additional information requested by the third party may also be provided from additional analysis of the stored raw data.

In some embodiments, the analysis provides information on, for example, disease presence, disease severity, subtype of disease, the presence and/or identity of multiple diseases and/or predisposition to diseases. In some embodiments, the analysis provides information from multiplexing different antibody tests, multiple analytes from the same disease, multiplexing tests for different diseases. In some embodiments, the assay is an antibody-antigen interaction assay, a peptide-peptide interaction assay, a peptide-protein interaction assay, a protein-protein interaction assay, or a kinase interaction assay. In some embodiments, the assay station is a fully or semi-automated robotic liquid handling station.

In some embodiments, after the test is complete, the raw data with no bar-code that never can be retraced is stored in a user-controlled database (one way storage). This non-retraceable raw data will be used to study the variability of the specific tests across populations and also see the correlation between different antigenic peptide analyte in designed-set to determine limits for the controls.

In some embodiments, a yearly subscription is provided to trend a set of key antigenic peptides representing different diseases to build a self-baseline for individual patients. In one embodiment of this method, the same person is tested on the same set of designed antigenic peptides that are biologically relevant or molecular mimicry, at different time frame to trend whether or not a predisposition or early detection of a diseases to identify the trend change with solid evidence of key disease related antigenic-peptides move from self-baseline and light up even to slightly higher level from the trend-range. Any improvements to the trending subscription based designed-set will always have the legacy of the earlier designed-set so the continuity to trend of the same person is not lost, but improved with new addition to reflect progress in diagnostics.

Unless specifically stated otherwise as apparent from the following discussion, it is appreciated that throughout the description, discussions utilizing terms such as “processing” or “computing” or “calculating” or “determining” or “displaying” or “analyzing” or “comparing” or “identifying” or the like, refer to the action and processes of a computer system, or similar electronic computing device, that manipulates and transforms data represented as physical (electronic) quantities within the computer system's registers and memories into other data similarly represented as physical (electronic) quantities within the computer system's registers and memories into other data similarly represented as physical quantities within the computer system memories or registers or other such information storage, transmission or display devices.

The present disclosure also relates to system apparatus for performing the operations herein. This apparatus may be specially constructed for the required purposes, or it may comprise a general purpose computer selectively activated or reconfigured by a computer program stored in the computer. Such a computer program may be stored in a computer readable storage medium, such as, but is not limited to, any type of disk including floppy disks, optical disks, CD-ROMs, and magnetic-optical disks, read-only memories (ROMs), random access memories (RAMs), EPROMS, EEPROMs, magnetic or optical cards, or any type of media suitable for storing electronic instructions, and each coupled to a computer system bus.

Various general purpose systems may be used with programs in accordance with the teachings herein, or it may prove convenient to construct more specialized apparatus to perform the required method procedures. The required structure for a variety of these systems will appear from the description below. In addition, the present disclosure is not described with reference to any particular programming language. It will be appreciated that a variety of programming languages may be used to implement the teachings as described herein.

### ADDITIONAL EMBODIMENTS

### Embodiment 1

A microarray comprising a surface and one or more features attached to the surface, wherein the features comprise one or more probe molecules from one or more tickborne pathogens.

### Embodiment 2

The microarray of embodiment, wherein the microarray is configured to have an at least 25%, 55%, 60%, 65%, 70%, 75%, 80%, 85%, 90%, 91%, 92%, 93%, 94%, 95%, 96%, 97%, 98%, 99%, or 100% sensitivity for detection of a tickborne pathogen after contact of the features with a sample from a subject suspected of having a tickborne pathogen.

### Embodiment 3

The microarray of embodiment or, wherein the microarray is configured to have an at least 90%, 91%, 92%, 93%, 94%, 95%, 96%, 97%, 98%, 99%, or 100% specificity for detection of a tickborne pathogen after contact of the features with a sample from a subject suspected of having a tickborne pathogen.

### Embodiment 4

The microarray of embodiments 1-, wherein the microarray is configured to have at least 90% sensitivity and 90% specificity for detection of a tickborne pathogen after contact of the features with a sample from a subject suspected of having a tickborne pathogen.

### Embodiment 5

The microarray of embodiments 1-, wherein the microarray is configured to have at least 93% sensitivity and 96% specificity for detection of a tickborne pathogen after contact of the features with a sample from a subject suspected of having a tickborne pathogen.

### Embodiment 6

The microarray of embodiments 1-, wherein the microarray is configured to have at least 95% sensitivity and 95% specificity for detection of a tickborne pathogen after contact of the features with a sample from a subject suspected of having a tickborne pathogen.

### Embodiment 7

The microarray of any one of embodiments-, wherein the one or more tickborne pathogens comprises Borrelia burgdorferi, Borrelia mayonii, Borrelia afzelii, Borrelia garinii, Borrelia Bavariensis, Borrelia speielmanii, Borrelia hemsii, Borrelia Turicatae, Borrelia miyamotoi, Borrelia andersonii, Borrelia maritima, Borrelia californiensis, Borrelia bissettiae, Borrelia lusitaniae, Borrelia valaisiana, Borrelia yangtzensis, Borrelia turcia, Borrelia lonestari, Babsia microti, Bartonella henselae, Anaplasma phagocytophilum, Ehrlichia chaffeensis, Rickettsia typhi, Rickettsia rickettsia, Rickettsia parkeri, Francisella tularensis, Powassan virus, Tick-borne encephalitis virus, West Nile virus, Coxsackie virus, Cytomegalovirus, Epstein Barr virus, Parvovirus B19, Toxoplasma, gondii, Colorado tick fever, heartland virus, rickettsiosis, Rocky Mountain spotted fever, tularemia, Q fever, HSV-1, HSV-2, HHV-6, or HHV-7.

### Embodiment 8

The microarray of embodiments-, wherein the one or more tickborne pathogens is selected from Tables 1, 3, or A.

### Embodiment 9

The microarray of any one of embodiments-, wherein the one or more tickborne pathogen comprises Borrelia burgdorferi.

### Embodiment 10

The microarray of any one of embodiments-, wherein the microarray comprises one or more probe molecules from distinct tickborne pathogens.

### Embodiment 11

The microarray of any one of embodiments-, wherein the microarray comprises one or more probe molecules from at least 1, 2, 3, 4, 5, 6, 7, 8, 9, 10, 11, 12, 13, 14, 15, 16, 17, 18 or more tickborne pathogens.

### Embodiment 12

The microarray of any one of embodiments-, wherein the microarray comprises one or more probe molecules from the same tickborne pathogen.

### Embodiment 13

The microarray of any one of embodiments-, wherein the array comprises at least 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 32, 330, 340, 350, 360, 370, 380, 390, 400 or more probe molecules from one or more tickborne pathogen.

### Embodiment 14

The microarray of any one of embodiments-, wherein the microarray comprises at least four hundred or more probe molecules from one or more tickborne pathogen.

### Embodiment 15

The microarray of any one of embodiments-, wherein the one or more probe molecules are distinct from each other.

### Embodiment 16

The microarray of any one of embodiments-, wherein the probe molecules comprise a lysate, antigens or antigenic fragments, proteins, or peptides.

### Embodiment 17

The microarray of claim one of embodiments-, wherein the probe molecules comprise at least one lysate, at least one protein, and at least one peptide.

### Embodiment 18

The microarray of any one of embodiments-, wherein the one or more probe molecules is selected from Table 3 or Table B.

### Embodiment 19

The microarray of any one of embodiments-, wherein the one or more probe molecules comprise:
a. B. burgdorferi VLsE1, C6 peptide, DbpB, OspC, p18, p32, p28, p30, p31, OspA, OspB, BmpA, p34, p39, P41, p45, p58, p66, p83, p84, p85 p86, p87, p88, p89, p90, p91, p92, p93, whole cell sonicate crude extract B31, or whole cell sonicate crude extract B297;
b. B. afzelii BmpA, DbpA, OspA, OscpC, or p100;
c. B. garinii DBpA or OspC;
d. B. bavariensis p58, VLsE1, DbpA;
e. B. spielmanii DBpA or OspC;
f. B. miyamotoi GlpQ;
g. Babersia microtia IRA, p32, p41,
h. Bartonella henselae 17 kDa, 26 kDa, or SucB;
i. Anaplasma phgocytophilum MSP5, MSP2, or OmpA;
j. Rickettsia typhi OmpB or surface antigen;
k. Cytomeglovirus EIA, gB, p150, p28, p52, pp65, or p38;
1. Epstein Barr virus EA, EBNA1, VCA, gp125, p18, or p23;
m. Parvovirus VLP VLP2, VLP VP1/VP2;
n. Toxoplasma gondii MIC3, p24, p29, or 30;
o. Rickettsia rickettsia rompA or rompB;
p. Rickettsia parkeri OmpA, OmpB, PS 120, or 17 kDa;
q. Francisella tularensis LPS O antigen;
r. Heartland virus Gn or Gc; and/or
s. lysate derived from one or more of Borrelia burgdorferi, Borrelia mayonii, Borrelia afzelii, Borrelia garinii, Borrelia Bavariensis, Borrelia speielmanii, Borrelia hemsii, Borrelia Turicatae, Borrelia miyamotoi, Borrelia andersonii, Borrelia maritima, Borrelia californiensis, Borrelia bissettiae, Borrelia lusitaniae, Borrelia valaisiana, Borrelia yangtzensis, Borrelia turcia, Babsia microti, Bartonella henselae, Anaplasma phagocytophilum, Ehrlichia chaffeensis, Rickettsia typhi, Rickettsia rickettsia, Powassan virus, Tick-borne encephalitis virus, West Nile virus, Coxsackie virus, Cytomegalovirus, Epstein Barr virus, Parvovirus B19, Toxoplasma, gondii, HSV-1, HSV-2, HHV-6, HHV-7, Colorado tick fever virus, Bourbon virus, or Borrelia lonestari.

### Embodiment 20

The microarray of any one of embodiments-, wherein the one or more probe molecules comprise B. burgdorferi VLsE1, C6 peptide, B31, B297, p18, p28, p30, p31, p34, p39, P41, 45, p58, p66, and p93.

### Embodiment 21

The microarray of any one of embodiments-, wherein the one or more tickborne pathogens is carried by at least one tick species selected from Table 3 or Table A.

### Embodiment 22

The microarray of any one of embodiments-, wherein the microarray comprises probe molecules from one or more tick species selected from Table 3 or Table A.

### Embodiment 23

The microarray of any one of embodiments-, wherein the micro array comprises probe molecules from at least 1, 2, 3, 4, 5, 6, 7, 8, 9, 10, 11, 12, 13, 14, 15, 16, 17, 18, 19, 20, or more tick species.

### Embodiment 24

The microarray of any one of embodiments-, wherein the tick species is any one of I. scpularis, I. ricinus, I. persulatus, I. uriae, O. hermsi, O. turicatae, I. dentatus, I. pacificus, I. spinipalpis, I. jellisonii, I. nippopensis, I. columnae, I. granulatus, H. aegypticum; A. Americanum, D. variabilis, D. andersoni, H. longicornis, I. cookie, O. moubata, Borrelia lonestari, Dermacentor variabilis, Dermacentor andersoni, Rhipicephalus sanguineus, Dermacentor similis, Amblyomma Americanum, Amblyomma maculatum, D. similis.

### Embodiment 25

The microarray of any one of embodiments-, wherein the tickborne pathogen generates an immune response in a subject.

### Embodiment 26

The microarray of any one of embodiments-, wherein the tickborne pathogen causes a disease in a subject.

### Embodiment 27

The microarray of claim or, wherein the subject is human.

### Embodiment 28

The microarray of any one of embodiments-, wherein the features are at positionally-defined locations on the surface.

### Embodiment 29

The microarray of any one of embodiments 1-, wherein the surface comprises one or more silicon wafer microchips.

### Embodiment 30

The microarray of any one of embodiments 1-, wherein the microarray is attached to a support surface.

### Embodiment 31

The microarray of embodiment, wherein the support surface is a pillar plate.

### Embodiment 32

The microarray of embodiment, wherein the pillar plate comprises a 12, 24, 36, 48, or 96 pillar plate.

### Embodiment 33

A method for obtaining feature binding data, comprising:
obtaining the microarray of any one of embodiments 1-,
contacting the microarray with a sample comprising a plurality of ligands for at least a subset of the features under conditions that promote ligand binding; and
imaging the microarray to identify binding of the plurality of ligands to the features of the microarray.

### Embodiment 34

A method of identifying a tickborne disease or pathogen in a subject, comprising:

contacting a sample from the subject with the microarray of any one of embodiments 1-; and

identifying binding of ligands in the sample to the features on the microarray to determine whether the subject has the tickborne disease or pathogen.

### Embodiment 35

The method of embodiment, wherein the identifying binding of ligands in the sample comprises:
contacting the microarray with a sample comprising a plurality of ligands for at least a subset of the features under conditions that promote ligand binding; and
imaging the microarray to identify binding of the plurality of ligands to the features of the microarray.

### Embodiment 36

The method of any one of embodiments-, wherein the tickborne disease or pathogen is Lyme disease, Borrelia burgdorferi, Borrelia mayonii, Borrelia afzelii, Borrelia garinii, Borrelia Bavariensis, Borrelia speielmanii, Borrelia hemsii, Borrelia Turicatae, Borrelia miyamotoi, Borrelia andersonii, Borrelia maritima, Borrelia californiensis, Borrelia bissettiae, Borrelia lusitaniae, Borrelia valaisiana, Borrelia yangtzensis, Borrelia turcia, Borrelia lonestari, Babsia microti, Bartonella henselae, Anaplasma phagocytophilum, Ehrlichia chaffeensis, Rickettsia typhi, Rickettsia rickettsia, Rickettsia parkeri, Francisella tularensis, Powassan virus, Tick-borne encephalitis virus, West Nile virus, Coxsackie virus, Cytomegalovirus, Epstein Barr virus, Parvovirus B19, Toxoplasma, gondii, Heartland virus, Colorado tick fever virus, HSV-1, or HHV-6.

### Embodiment 37

The method of any one of embodiments-, wherein the sample is from a subject.

### Embodiment 38

The method of any one of embodiments-, wherein the sample is blood or serum.

### Embodiment 39

The method of any one of embodiments-, wherein the subject is human.

### Embodiment 40

The method of any one of embodiments-39, wherein the ligands comprise an antibody or antigen-binding fragment.

### Embodiment 41

The method of embodiment, wherein the ligand comprises an IgG antibody or IgM antibody or a combination thereof.

### Embodiment 42

The method of any one of embodiments-, wherein the method comprises identifying binding of IgG, IgM or IgG and IgM antibodies present in the sample to the probe molecules on the microarray.

### Embodiment 43

The method of any one of embodiments-, wherein the method comprises a sensitivity of detection of IgM antibodies that bind to the tickborne disease or pathogen with a sensitivity of at least 25%, 55%, 60%, 65%, 70%, 75%, 80%, 85%, 90%, 91%, 92%, 93%, 94%, 95%, 96%, 97%, 98%, or 99%.

### Embodiment 44

The method of any one of embodiments-, wherein the method comprises a specificity of detection of IgM antibodies that bind to the tickborne disease or pathogen with a sensitivity of at least 95%, 96%, 97%, 98%, 99%, or 100%.

### Embodiment 45

The method of any one of embodiments-, wherein the method comprises a sensitivity of detection of IgG antibodies that bind to the tickborne disease or pathogen with a sensitivity of at least 80%, 83%, 85%, 87%, 90%, 91%, 92%, 93%, 94%, 95%, 96%, 97%, 98%, 99%, or 100%.

### Embodiment 46

The method of any one of embodiments-, wherein the method comprises a specificity of detection of IgM antibodies that bind to the tickborne disease or pathogen with a sensitivity of at least 95%, 96%, 97%, 98%, 99%, or 100%.

### Embodiment 47

The method of any one of embodiments-, wherein the method comprises a sensitivity of detection of IgG and IgM antibodies that bind to the tickborne disease or pathogen with a sensitivity of at least 85%, 87%, 90%, 91%, 92%, 93%, 94%, 95%, 96%, 97%, 98%, 99%, or 100%.

### Embodiment 48

The method of any one of embodiments-, wherein the method comprises a specificity of detection of IgG and IgM antibodies that bind to the tickborne disease or pathogen with a sensitivity of at least 95%, 96%, 97%, 98%, 99%, or 100%.

### Embodiment 49

The method of any one of embodiments-, wherein a total number of features on the microarray is at least about 100, 200, 300, 400, 500, 600, 700, 800, 900, or 1000.

### Embodiment 50

The method of embodiments-, wherein the microarray has an area that is less than or equal to 0.2 square millimeters (mm²), less than or equal to 0.7 square millimeters (mm²), less than or equal to 1 square millimeters (mm²), less than or equal to 10 square millimeters (mm²), less than or equal to 100 square millimeters (mm²), or less than or equal to 150 square millimeters (mm²).

### Embodiment 51

The method of any one of embodiments-, wherein the sample has a volume that is less than or equal to 100 μL, less than or equal to 50 μL, less than or equal to 25 μL, less than or equal to 10 μL, less than or equal to 5 μL, less than or equal to 1.5 μL, or less than or equal to 1 μL.

### Embodiment 52

The method of any one of embodiments-, wherein an elapsed time from sample contacting to finishing the imaging is equal or less than 20 minutes, equal or less than 5 minutes, equal or less than 1 minute, equal or less than 2-seconds, equal or less than 10 seconds, equal or less than 1 second.

### Embodiment 53

The method of any one of embodiments-, wherein a coefficient of variation of data obtained from the array is not greater than 5 percent, not greater than 2 percent, not greater than 1 percent.

### Embodiment 54

The method of any one of embodiments-, wherein the microarray comprises at least at least 1000 features per square centimeter, at least 5,000 features per square centimeter, at least 10,000 features per square centimeter, at least 50,000 features per square centimeter, at least 100,000 features per square centimeter, at least 500,000 features per square centimeter, at least 1,000,000 features per square centimeter, at least 10,000,000 features per square centimeter, or at least 15,000,000 features per square centimeter.

### Embodiment 56

The method of any one of embodiments-, the contacting occurs at a concentration of the plurality of ligands that is less or equal than 1,000 μg/ml in the sample, less or equal than 10 μg/ml in the sample, less or equal than 1 μg/ml in the sample, less or equal than 0.1 μg/ml in the sample, less or equal than 10 ng/ml in the sample, less or equal than 1 ng/ml in the sample, less or equal than 5 μg/ml in the sample.

### Embodiment 57

The method of any one of embodiments-, the contacting occurs at a concentration of the plurality of ligands that is within the range of approximately 1 μg/ml to approximately 1,000 μg/ml in the sample.

### Embodiment 58

The method of any one of embodiments-, wherein the imaging comprises identifying binding of at least 100 ligands, at least 500 ligands, at least 1,000 ligands, at least 100,000 ligands, at least 1,000,000 ligands, at least 10,000,000 ligands, at least 15,000,000 ligands, or at least 100,000,000 ligands to the features of the microarray.

### EXAMPLES

Below are examples of specific embodiments for carrying out the present disclosure The examples are offered for illustrative purposes only, and are not intended to limit the scope of the present disclosure in any way. Efforts have been made to ensure accuracy with respect to numbers used (e.g., amounts, temperatures, etc.), but some experimental error and deviation should, of course, be allowed for.

The practice of the present disclosure will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W. H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pennsylvania: Mack Publishing Company, 1990); Carey and Sundberg Advanced Organic Chemistry 3rd Ed. (Plenum Press) Vols A and B (1992).

### Example 1: Tickborne Microarray Development and Use

Most vector-borne infections in the USA can be attributed to pathogens transmitted via tick bites. Of all tick-borne infections identified to date, Lyme disease is the most prevalent infection. Lyme disease is a potentially serious bacterial infection transmitted by ticks and was first reported in the mid-1970s in the USA. The etiological agent was identified later as Borrelia burgdorferi. Co-infections can be present with Lyme disease including Babesia spp., Bartonella spp., Ehrlichia spp., Anaplasma phagocytophilum, Powassan virus (POWV), Toxoplasma gondii, Rickettsia spp., tick-borne encephalitis virus (TBEV), and West Nile virus (WNV). Additionally, prolonged exposure to Lyme and other tick-borne infections can weaken a subject's immune system increasing the risk of infections like Epstein Barr virus (EBV), cytomegalovirus (CMV), parvovirus B19 (B19V), coxsackie B virus, herpes simplex virus 1 (HSV-1), herpes simplex virus 2 (HSV-2), and human herpes virus 6 (HHV-6).

Ticks have been shown to transmit more than one infectious agent in a single bite. For instance, there is a risk of getting infected with A. phagocytophilum (30%) and B. microti (24%) along with B. burgdorferi, Wormser, G. P., et al (2019). Emerging infectious diseases, 25(4), 748. Currently, multi-tiered testing is carried out for diagnosing tick-borne infections (Quest diagnostics. (2018). Tick-borne Diseases. Quest Diagnostics). In this method, the infectious agents are tested sequentially, starting with B. burgdorferi. This method is time-consuming and can often lead to delayed diagnosis, accompanied with high cost to the patient. Testing for multiple infections in a single run can help physicians arrive at an accurate diagnosis especially since Lyme disease shares symptoms with other vector-borne co-infections. The existing diagnostic assays possess various limitations that restrict their applicability in the diagnosis of these infections. The diagnosis of Lyme disease and other infectious diseases using several blot-based and single-plex ELISA tests remain rudimentary in terms of arriving at a diagnostic conclusion. Additionally, blot-based assays may have overlapping proteins with similar mass requiring additional testing to tease out the specific antigen to which the antibody is bound. A multiplex system can detect the biomarkers of Lyme disease, potential co-infections, and other infections in a single run. A serology-based multiplexing system may be preferred to a PCR multiplex system mainly due to its accessibility, for instance using dried blood spots (DBS). Due to the transient nature of the organisms, the timeline in which the patients are bacteremic, viremic, and/or parasitemic is short making it difficult to obtain genetic material of the pathogens for nucleic acid-based diagnosis. However, this limitation is overcome by using serology. A serology-based system is also ideal for population screening and surveillance since it can indicate past exposure to a pathogen.

A multiplex protein microarray was designed to detect multiple serological antibodies thereby detecting exposure to multiple pathogens in a single run. The microarray can accommodate at least 400 antigens and can multiplex across antigen types, lysates, recombinant proteins, and peptides. A designed array containing multiple antigens of several tick-borne microbes including Borrelia burgdorferi, the Lyme disease spirochete, was manufactured and evaluated, additionally antigens for several infections were also validated. The immunoglobulin M (IgM) and G (IgG) responses against several tick-borne microbes and other infectious agents were analyzed for analytical and clinical performance. The microarray improved IgM and IgG sensitivities and specificities of individual microbes when compared with the respective gold standards. The testing was also performed in a single run in comparison to multiple runs needed for comparable testing standards. In summary, the present study presents a multiplex microarray platform that can provide quick results with high sensitivity and specificity for evaluating exposure to varied infectious agents especially tickborne infections.

Tick pathogens included in the panel were Bartonella henselae, Ehrlichia chaffeensis, Anaplasma phagocytophilum, Borrelia burgdorferi, Rickettsia typhi, tick-borne encephalitis virus, Parvovirus B19, West Nile virus, Coxsackie group B virus, Powassan virus, Epstein Barr Virus, cytomegalovirus, herpes simplex virus 1, human herpes virus type 6, Toxoplasma gondii and Babesia microti. The diagnostic tests, including gold standards (Table A) available for these pathogens are severely limited in their ability to test for them accurately.

### Materials and Methods

### Patients Sera

The sera from 2990 individuals were collected after seeking appropriate IRB approval under respective collaborators. Table 1 lists the provided samples for Lyme disease, co-infections, and other infections along with the counts, respective collaborators and methods used to ascertain the clinical diagnosis by the physician. These reference sera were tested at Vibrant America Clinical Labs (CLIA and CAP accredited facility) by laboratory personnel in a blinded manner. The sera from healthy patients were considered negative and were used to set the cut-off values and were investigated under IRB exemption (work order #1-1098539-1) determined by the Western Institutional Review Board (WIRB) to employ de-linked and de-identified human specimens and medical data for research findings. The negative sera were collected from across the US including endemic and nonendemic regions for these infections. Table 2 provides the vendor and pathogen strain information used in the study.

TABLE 1
Sample Cohort

| Pathogen | N | Source (scientist/company) | Basis of Diagnosis |
|---|---|---|---|
| Borrelia burgdorferi | 298 | CDC, Private Clinics | Physician |
| Babesia microti | 70 | Susan Madison-Antenucci | RT-PCR, Blood Smear |
| | | (NYSDOH) | |
| Babesia microti | 118 | Seracare, Boca Bio, Private | Serology, RT-PCR |
| | | Clinics | |
| Babesia microti | 26 | Renata Welc-Falȩciak | Physician |
| | | (University of Warsaw), | |
| | | Agnieszka Pawełczyk (Warsaw | |
| | | Medical University) | |
| Bartonella henselae | 119 | Private clinics | Physician |
| Bartonella henselae | 26 | Renata Welc-Falȩciak | Physician |
| | | (University of Warsaw), | |
| | | Agnieszka Pawełczyk (Warsaw | |
| | | Medical University) | |
| Bartonella henselae | 10 | Dimosthenis Chochlakis | Physician |
| | | (University of Crete) | |
| Anaplasma phagocytophilum | 118 | Private clinics, Boca Bio | Serology |
| Anaplasma phagocytophilum | 26 | Renata Welc-Falȩciak | Physician |
| | | (University of Warsaw), | |
| | | Agnieszka Pawełczyk (Warsaw | |
| | | Medical University) | |
| Ehrlichia chaffeensis | 120 | Private clinics, Boca Bio | Serology |
| Ehrlichia chaffeensis | 26 | Renata Welc-Falȩciak | Physician |
| | | (University of Warsaw), | |
| | | Agnieszka Pawełczyk (Warsaw | |
| | | Medical University) | |
| Rickettsia typhi | 70 | Lucas Blanton (University of | Physician |
| | | Texas Medical Branch) | |
| Rickettsia typhi | 124 | Private clinics | Physician |
| Rickettsia rickettsii | 21 | Angélica Escárcega-Ávila | Physician |
| | | (Autonomous University of | |
| | | Ciudad Juarez) | |
| Rickettsia rickettsii | 124 | Private clinics | RT-PCR, Physician |
| Powassan virus | 127 | Private clinics | Physician |
| Tick-borne encephalitis | 111 | Daniel Rużek (Czech Academy | Physician |
| | | of Sciences) | |
| Tick-borne encephalitis | 124 | Private clinics | Physician |
| West Nile virus | 20 | Gheyath K. Nasrallah (Weill | Physician |
| | | Cornell Medicine-Qatar) | |
| West Nile virus | 124 | Private clinics | Physician |
| Coxsackie virus | 45 | iSpecimen | Serology |
| Coxsackie virus | 124 | Private clinics | Physician |
| Cytomegalovirus | 43 | DLS | Serology |
| Cytomegalovirus | 138 | Daniele Lilleri (Fondazione | Physician |
| | | IRCCS Policlinico San Matteo) | |
| Cytomegalovirus | 96 | Private Clinics | Physician |
| Cytomegalovirus | 37 | Seracare | Serology |
| Epstein Barr virus | 20 | Gheyath K. Nasrallah (Weill | Physician |
| | | Cornell Medicine-Qatar) | |
| Epstein Barr virus | 43 | Seracare | Serology |
| Epstein Barr virus | 14 | iSpecimen | Serology |
| Epstein Barr virus | 96 | Private clinics | Physician |
| Parvovirus B19 | 99 | Maria Söderlund-Venermo | Serology |
| | | (University of Helsinki) | |
| Parvovirus B19 | 124 | Private clinics | Physician |
| Toxoplasma gondii | 24 | Seracare | Serology |
| Toxoplasma gondii | 124 | Private clinics | Physician |
| HSV-1 | 20 | Gheyath K. Nasrallah (Weill | Physician |
| | | Cornell Medicine-Qatar) | |
| HSV-1 | 31 | Seracare | Serology |
| HSV-1 | 96 | Private clinics | Physician |
| HSV-2 | 19 | Gheyath K. Nasrallah (Weill | Physician |
| | | Cornell Medicine-Qatar) | |
| HSV-2 | 27 | Seracare | Serology |
| HSV-2 | 96 | Private clinics | Physician |
| HHV-6 | 20 | Gheyath K. Nasrallah (Weill | Physician |
| | | Cornell Medicine-Qatar) | |
| HHV-6 | 96 | Private clinics | Physician |

TABLE 2
Pathogen strains and vendors

| Name | Vendor | Strain ID |
|---|---|---|
| B. burgdorferi B31 | ATCC | 35210 |
| B. burgdorferi 297 | ATCC | 53899 |
| B. mayonii | ATCC | BAA-2743 |
| B. hermsii | ATCC | BAA-2821 |
| B. andersoni | ATCC | 700555 |
| B. maritima | ATCC | TSD-160 |
| B. californiensis | DSMZ | DSM 17989 |
| B. bissettiae | DSMZ | DSM 17990 |
| B. lusitaniae | DSMZ | DSM 107168 |
| B. valaisiana | DSMZ | DSM 21467 |
| B. yangtzensis | DSMZ | DSM 24625 |
| B. turcica | DSMZ | DSM 16138 |
| Babesia microti | ATCC | PRA-99 |
| Babesia duncani | ATCC | PRA-302 |
| Bartonella elizabethae | ATCC | 49927 |
| Bartonella vinsonii | DSMZ | DSM 28218 |
| Bartonella quintana | ATCC | 51694 |
| Ehrlichia chaffeensis | ATCC | CRL-10389 |
| HHV-6A | ZeptoMetrix | 0810529CF |
| HHV-6B | ZeptoMetrix | 0810072CF |
| HHV-7 | ZeptoMetrix | 0810071CF |
| T. gondii | Biospacific | J46520 |

### Processing of Wafers

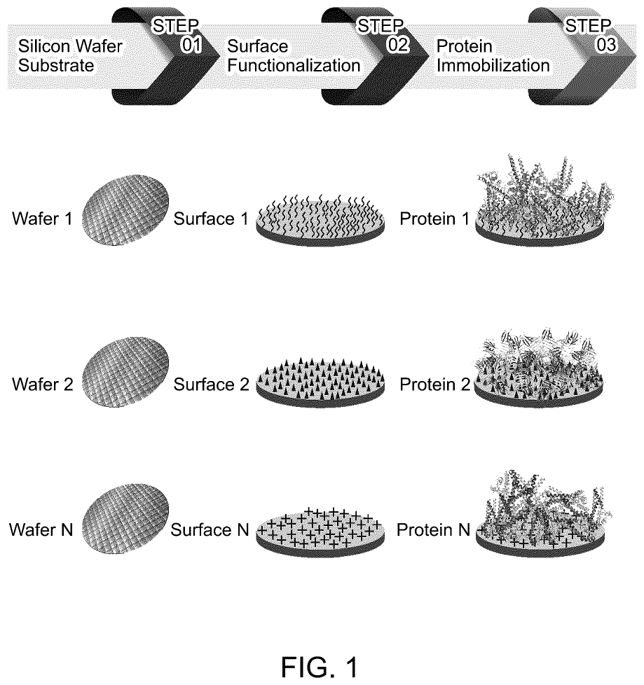

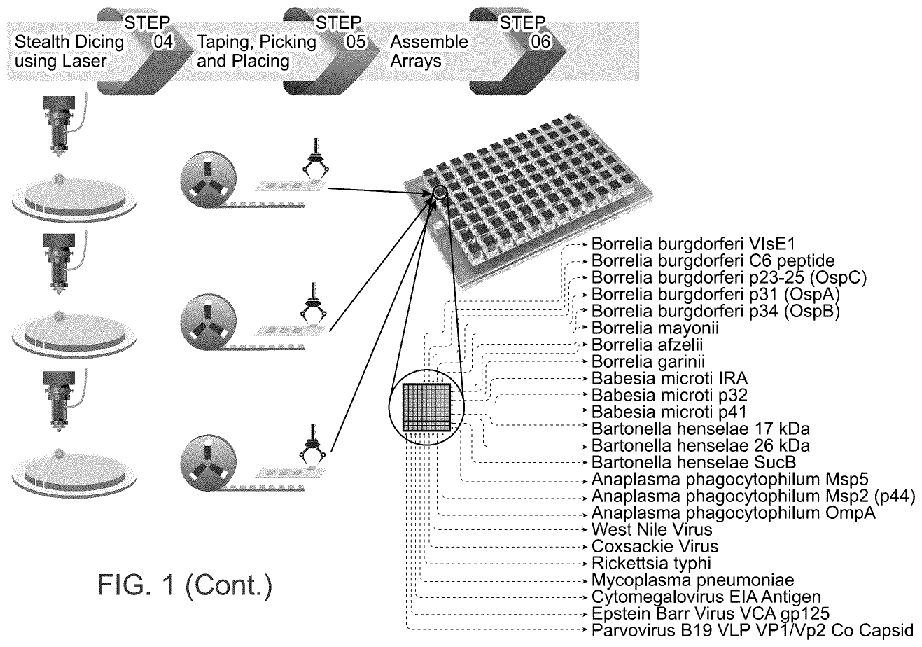

Wafers were functionalized as described in U.S. Pat. No. 9,417,236. Briefly, silicon wafers were exposed to an environment of pure oxygen for 2 h followed by washing (deionized Water) and coating (1% (vol/vol) of 3-aminopropyltriethoxysilane (APTES) in N-methylpyrrolidone (NMP). Curing was carried out at 120° C. for 60 min under an N₂ atmosphere and humidity-controlled environment. Coating and incubation of the wafer with a co-polymer solution of poly(L-lysine) and poly(lactic acid) for 24 h were carried out to increase the binding efficiency of the surface on to which the antigens were immobilized via passive adsorption/hydrophobic interactions with the copolymers (FIG. 1).

### Immobilization of Antigens

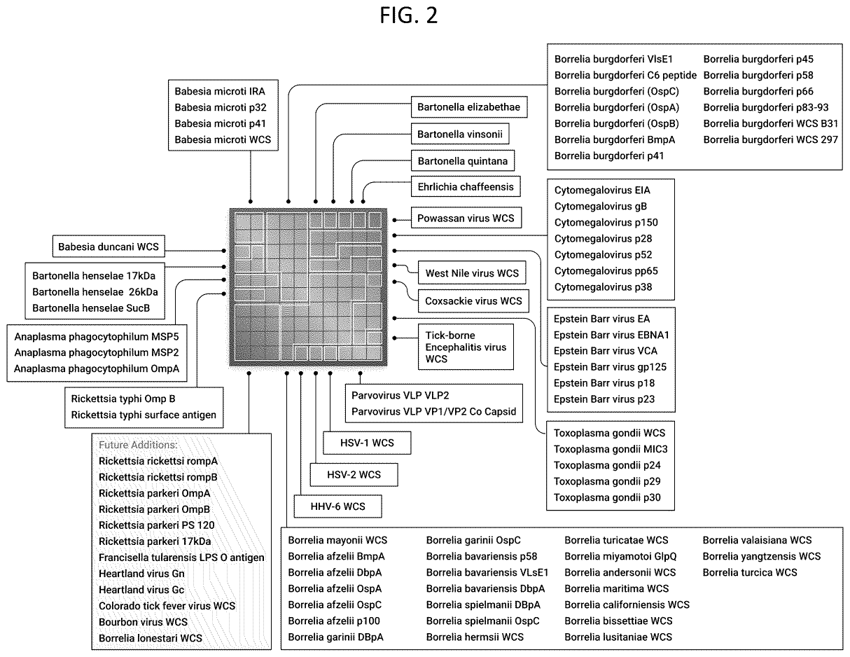

The antigens included in the microarray assay are listed in Table 3. The recombinant antigens were expressed in E. coli bacteria using full-length cDNA coding for the respective antigens fused with a hexa histidine purification tag. The whole cell lysate was obtained from organisms cultured according to ATCC protocols prior to lysing them which yielded a cocktail of the cell membrane, cell wall, and cytosolic proteins. Peptide antigens were synthesized by photolithography as previously described in Marietta, E. V. et al (2016). PLOS One, 11(1), e0147777 and Rostamkolaei, S. K et al. (2019) Gastroenterology, 156 (3), 582-591.el, both of which are hereby incorporated by reference in their entirety. The capture antigens including the recombinant antigens that mimic the natural pathogen and the whole-cell sonicate were incubated on the wafer at a concentration of 1.0 μg/ml and reacted for 24 h at 4° C. The unbound antigens were removed by washing with aqueous phosphate buffer and the unreacted substrate was quenched with a blocking solution containing BSA and glycine. The immobilized antigens were classified with unique identifiers assigned to each wafer. The microarray was configured to detect Lyme disease, co infections and other infections including B. microti, B. henselae, A. phagocytophilum, E. chaffeensis, R. typhi, R. rickettsii, Powassan virus (POWV), Tick-borne encephalitis virus (TBEV), West Nile virus (WNV), Coxsackie B virus, Cytomegalovirus (CMV), Epstein Barr virus (EBV), Parvovirus B19 (B19V), T. gondii, HSV-1, HSV-2, and HHV-6 (FIG. 2).

TABLE 3
Pathogens and antigens

| Pathogen (Tick, if any) | Cell Lysate/Peptide | Recombinant Antigen(s) |
|---|---|---|
| B. burgdorferi (I. scapularis) | Whole cell lysate - B31 | VlsE1, DbpB, OspC, p28, p30, |
| | strain, 297 strain. | OspA, OspB, BmpA, p41, p45, p58, |
| | C6 peptide | p66, p83-93 |
| B. mayonii (I. scapularis) | Whole cell lysate | |
| B. afzelii (I. ricinus, I. persulatus) | | BmpA, DbpA, OspA, OspC, p100 |
| B. garinii (I. ricinus, I. persulatus) | | DBpA, OspC |
| B. bavariensis (I. uriae, I. | | p58, VIsEl, DbpA |
| persulcatus) | | |
| B. spielmanii (I. ricinus) | | DBpA, OspC |
| B. hermsii (O. hermsi) | Whole cell lysate | |
| B. turicatae (O. turicatae) | Whole cell lysate | |
| B. miyamotoi (I. dentatus, I. ricinus, | | GlpQ |
| I. scapularis, I. pacificus) | | |
| B. andersonii (I. dentatus) | Whole cell lysate | |
| B. maritima (I. spinipalpis) | Whole cell lysate | |
| B. californiensis (I. jellisonii, I. | Whole cell lysate | |
| spinipalpis, I. pacificus) | | |
| B. bissettiae (I. scapularis, I. | Whole cell lysate | |
| persulatus, I. spinipalpis, I. | | |
| pacificus) | | |
| B. lusitaniae (I. ricinus) | Whole cell lysate | |
| B. valaisiana (I. ricinus, I. | Whole cell lysate | |
| nippopensis, I. columnae) | | |
| B. yangtzensis (I. granulatus, I. | Whole cell lysate | |
| nipponensis) | | |
| B. turcica (H. aegypticum) | Whole cell lysate | |
| Babesia microti (I. ricinus, I. | Whole cell lysate | IRA, p32, p41 |
| scapularis, blood transfusions, | | |
| perinatal) | | |
| Babesia duncani (I. ricinus, I. | Whole cell lysate | |
| scapularis, blood transfusions, | | |
| perinatal) | | |
| Bartonella henselae | | 17 kDa, 26 kDa, SucB |
| Bartonella elizabethae | Whole cell lysate | |
| Bartonella vinsonii | Whole cell lysate | |
| Bartonella quintana | Whole cell lysate | |
| Anaplasma phagocytophilum (I. | | MSP5, MSP2, OmpA |
| scapularis, I. ricinus) | | |
| Ehrlichia chaffeensis (Amblyomma | Whole cell lysate | |
| americanum) | | |
| Rickettsia typhi (Flea Xenopsylla | | OmpB, surface antigen |
| cheopis, Ctenocephalides felis)) | | |
| Rickettsia rickettsii (Dermacentor | | OmpB, surface antigen |
| variabilis, Rhipicephalus | | |
| sanguineus, and Dermacentor | | |
| andersoni) | | |
| Powassan virus (Hemaphysalis | | NS1 |
| longicornis, I. scapularis, I. cookei) | | |
| Tick-borne encephalitis virus (I. | | NS1 |
| ricinus, I. persulcatus) | | |
| West Nile virus (I. ricinus, O. | | NS1 |
| moubata) | | |
| Coxsackie B virus (Amblyomma | | VP1 |
| americanum) | | |
| Cytomegalovirus | | EIA, GlyB, p150, p28, p52, pp65, |
| | | p38 |
| Epstein Barr virus | | EA, EBNA1, VCA gp125, p18, p23 |
| Parvovirus B19 | | VLP VLP2, VLP VP1/VP2 Co |
| | | Capsid |
| Toxoplasma gondii (multiple ticks) | Whole cell lysate | MIC3, p24, p29, p30 |
| HSV-1 | | gG-1 |
| HSV-2 | | gG-2 |
| HHV-6 | Whole cell lysate | |

### Pillar Plate Assembly

Individual wafers were stealth diced into 0.70×0.70 mm² microchips for each antigen. A standard die-sorting system was used to pick and place these wafers onto individual carrier tapes. The carrier tapes were then placed onto a high-throughput surface mount technology (SMT) component placement system. Finally, microchips were mounted onto 24-pillar plates. Each pillar contained a microarray of 87 microchips with each chip designated for one antigen derived from a recombinant protein, peptide or whole cell sonicate. A schematic of the microarray assembly is provided in FIG. 1 and FIG. 2.

### Immunochip Assay and Antibody Detection

Serum samples were probed using 1:20 dilution on the pillar plate and incubated for 1 h at room temperature followed by alternate washing and incubation as described previously Jayaraman, V et al, (2020) Scientific Reports, 10, 18085, hereby incorporated by reference in its entirety. The plate was then incubated for an hour with the secondary antibody (1:2000 dilution of Goat Anti-Human IgG HRP and Goat Anti-Human IgM HRP individually) and washed with TBST buffer followed by DI Water. The plates were left for drying preceding the addition of chemiluminescent substrate and the performance of chemiluminescent imaging. An enhanced IgM sensitivity was achieved by pre-reacting the sera with proprietary assay components leading to IgG stripping prior to IgM testing.

The detection of multiplex antibodies was performed by chemiluminescent immunoassay and can be performed using <100 μL of serum. Sample dilution, multi-step incubation, and multi-solution washing were programmed into liquid handlers. The immunochip had the capacity to assay 192 individual specimens in 2 h. Raw chemiluminescent signals for each probe were extracted and converted into intensity plots by an in-house reporter software. This method of automatic antigen detection can dramatically shorten the turnaround time, reduce the cost of labor and instrument, and eliminate the need for manual handling and subjective interpretation of the WB or IB test results when compared to the traditional two-tiered testing recommended by the CDC. All the antibodies were detected in a single run.

### Data Analysis

An in-house software extracts the chemiluminescent signals from the generated images which were converted to intensity plots. The average intensity of each antibody was compared with the cut-off values assigned for each antigen to track seropositivity.

### Results

### Custom Protein Microarray Platform

The main components of the Immunochip platform include multiple silicon-based 0.70×0.70 mm\hat{ }2 microchips that were laser diced from antigen-immobilized wafers, a customized 24-well compatible plate containing 24 pillars, each containing 87 microchips that were picked and placed into a multiplex microarray assembly, and a high-resolution imager capable of simultaneously detecting chemiluminescent signals from labelled antigen-antibody reactions at each microchip throughout the multiplex microarray (FIG. 1). Each chip is analogous to an individual band in a Western blot; however, the proteins are physically separated eliminating cross-reactive issues seen in blot-based assays for proteins with similar mass. FIG. 1 provides an overview of the microarray manufacturing process. FIG. 2 shows the individual chips that are placed in each pillar. A single serum sample is applied to each pillar thereby assaying the antibodies in serum against all antigens at the same time.

### Analysis of Serological Response

The novel tick-borne disease panel tested for IgG and IgM antibodies for Lyme disease and other infectious agents as described in Table 2 and Table 3. The IgM and IgG immune responses of serum samples obtained from various laboratories (Table 1) were analyzed and the clinical sensitivity and specificities tabulated (Table 4). The samples reacted with specific immunoreactive epitopes on the 87 different antigens tested. The immunoreactivity of these antigens was contrasted with that of the controls.

Cross Reactivity within Borrelia and Bartonella

### Enhanced IgM Assay

IgM antibodies are markers of an acute primary infection and are produced during the first two weeks of infection, whereas IgG antibodies are produced few days later and may remain for life. Total IgM antibodies make up only 5% to 10% of all the circulating antibodies. However, B. burgdorferi IgM antibodies may persist in Lyme disease patients' years after the initial infection. An IgM assay was developed that included removal of most IgG antibodies and other non-specific proteins from the serum prior to the IgM immunoassay. IgG antibodies and other non-specific proteins were removed from the serum prior to the immunoassay. This increased the sensitivity and specificity of the assay. Human IgG was removed by incubating the serum with a purified goat anti-human (GAH) IgG Fc fragment and proprietary assay reagents.

### Analytical Performance

The analytical performance of the immunochip was evaluated for precision (repeatability/reproducibility), analytical sensitivity, reportable range, linearity and matrix equivalency studies. Samples for negatives, low or moderate positives, and high positives were run with duplication to determine the analytical performance metrics. The precision study used a panel of 11 samples and was run over a period of 20 days with 2 duplicates per run and 4 runs per day. The assay measured the range ten times within the same run to determine simple precision whereas complex precision was determined by assaying two replicates of these samples twice daily over five days (see Appendix A of U.S. application 63/537,449 incorporated by reference in its entirety). Lot to Lot reproducibility was also tested to check for variation in manufacturing of the pillar plates by running a panel of 11 samples with 5 replicates per run, 3 runs per day over a period of 5 days using 3 manufactured lots, and tabulated (see Appendix B of U.S. application 63/537,449 incorporated by reference in its entirety). Analytical sensitivity was determined by running protein-free serum matrix samples and low antibody concentration samples, two replicates per run with two runs per day over a period of three days. The limit of blank (LoB) and limit of quantitation (LoQ) was calculated using the mean and standard deviation of the blank and the low antibody concentration samples (see Appendix C of U.S. application 63/537,449 incorporated by reference in its entirety). The linearity and reportable range were verified by running samples with varying level of antibodies and checking assay recovery (see Appendix D of U.S. application 63/537,449 incorporated by reference in its entirety). Matrix equivalence studies were also performed (see Appendix E of U.S. application 63/537,449 incorporated by reference in its entirety). The potential interference of specific endogenous and exogenous substances with the immunochip was evaluated by performing an interfering substance study. The interfering substances tested were 60 mg/dl bilirubin, 100 mg/ml cholesterol, 1000 mg/ml triglycerides, 1000 mg/ml hemoglobin, 6 g/dl albumin, and 3000 U/L Heparin. There was no interference between the immunochip and the substances tested at the mentioned levels.

### Clinical Sensitivity and Specificity

Table 4 provides an overview of the IgG and IgM sensitivities measured by the microarray. This is compared with the sensitivities and specificities of the current gold standard tests for the particular pathogen indicated. The microarray was able to achieve high sensitivities and specificities when compared with the gold standards for each pathogen. Table A provides details on the gold standard diagnostic tests for the pathogens along with the modes of transmission and their endemic regions.

TABLE 4
Antigen sensitivities

| | IgM | | IgG | | IgM + IgG | | Gold Std | | |
|---|---|---|---|---|---|---|---|---|---|
| | Sensitivity | Specificity | Sensitivity | Specificity | Sensitivity | Specificity | | Sensitivity | Specificity |
| Pathogen | (%) | (%) | (%) | (%) | (%) | (%) | | (%) | (%) |
| Borrelia | 60 | 100 | 94 | 100 | 100 | 100 | Two-tiered | Early: 77.6 | 95-100 |
| burgdorferi | | | | | | | test | Late: 70-100 | |
| Babesia microti | 79 | 98 | 91 | 97 | 99 | 96 | blood smear | 70-80 | 94-100 |
| | | | | | | | exam | | |
| Bartonella | 81 | 99 | 96 | 99 | 100 | 98 | IFA | 100 | 96.8 |
| henselae | | | | | | | | | |
| Anaplasma | 94 | 100 | 100 | 100 | 100 | 100 | PCR | 80-100 | 95-100 |
| phagocytophilum | | | | | | | | | |
| Ehrlichia | 84 | 100 | 100 | 99 | 100 | 99 | PCR | 83 | 100 |
| chaffeensis | | | | | | | | | |
| Rickettsia typhi | 86 | 100 | 83 | 100 | 97 | 100 | Sero assay | 83-100 (IFA IgG) | 91-100 (IFA IgG) |
| | | | | | | | | 53-100 (IFA IgM) | 91-99 (IFA IgM) |
| Rickettsia | 43 | 99 | 71 | 99 | 86 | 98 | Sero assay | 85-100 (IFA IgG) | 99-100 (IFA IgG) |
| rickettsii | | | | | | | | 83-85 (IFA IgM) | 100 (IFA IgM) |
| Powassan virus | 100 | 100 | 100 | 100 | 100 | 100 | Sero assay | 89 | 65(IFA IgG), |
| | | | | | | | | | 100 (IgM IFA) |
| Tickborne | 78 | 99 | 92 | 99 | 93 | 98 | Sero assay | 83-94 (IgG) | 100 (IgM) |
| encephalitis | | | | | | | | 94-100 (IgM) | 59-89 (IgG) |
| West Nile virus | 25 | 100 | 80 | 100 | 85 | 100 | Sero assay | 100 | 70.3 |
| Coxsackie virus | 56 | 100 | 100 | 100 | 100 | 100 | PCR | 94-97 | 100 |
| Cytomegalovirus | 100 | 100 | 100 | 100 | 100 | 100 | PCR, sero | 89.2 | 95 |
| Epstein Barr | 100 | 100 | 96 | 100 | | | Sero assay | 71 (VCA IgG), | 99 |
| virus VCA | | | | | | | | 95 (VCA IgM) | |
| Epstein Barr | 95 | 100 | 96 | 100 | 97 | 100 | 99 | 99 | 95 |
| virus EBNA1 | | | | | | | | | |
| Parvovirus B19 | 90 | 99 | 98 | 98 | 99 | 97 | PCR | >90 | >90 |
| Toxoplasma | 100 | 99 | 100 | 100 | 100 | 99 | Sero assay | 92.7 | 100 |
| gondii | | | | | | | | | |
| HSV-1 | 100 | 100 | 98 | 100 | 98 | 100 | Sero assay | 97-100 | 98 |
| HSV-2 | 100 | 100 | 98 | 100 | 98 | 100 | Sero assay | 97-100 | 98 |
| HHV-6 | 95 | 100 | 95 | 100 | 95 | 100 | rtPCR | >95 | >95 |

Evaluating the Antigens of Borrelia burgdorferi

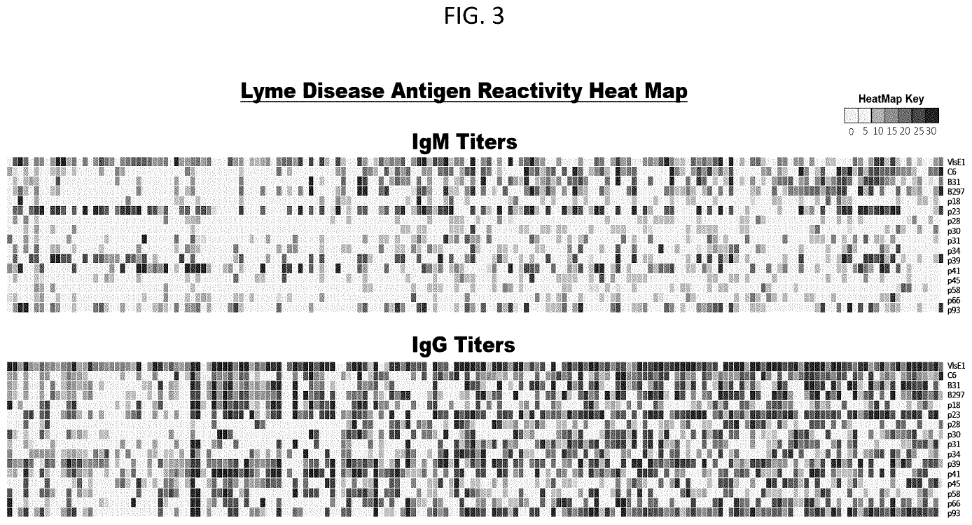

Individual antigens of B. burgdorferi was tested for reactivity with IgG and IgM antibodies (Table 5). The heat map (FIG. 3) shows the performance metrics of the different antigens. Testing for Lyme disease since 1994 has been based on conventional two-tiered testing (CTTT) where an enzyme immunoassay (EIA) is followed by a specific immunoblot for a definitive diagnosis. Recently, CTTT has been replaced with a modified two-tier testing (MTTT) in which an EIA using whole-cell sonicate is followed by an EIA using C6 peptide. This method has dropped the use of an immunoblot. MTTT removes the burden of immunoblots which are tedious to run, more expensive, and could have subjective interpretation of bands. Complete replacement of immunoblots can be done using a microarray platform such as the one described herein. The microarray developed here can detect all the antigens in a single run with a low sample volume. Testing for all the surface antigens on the Lyme spirochete is important to ensure a better diagnosis. A more complete data set would be available to physicians to make a nuanced diagnosis instead of a data set comprising only a narrow subset of antigens run on ELISAs.

TABLE 5
Individual antigen sensitivities (B. burgdorferi)

| Antigen | IgM | IgG |
|---|---|---|
| VlsE1 | 61% | 91% |
| C6 | 39% | 60% |
| B31 | 37% | 48% |
| B297 | 36% | 57% |
| p18 | 6% | 41% |
| p23 | 47% | 68% |
| p28 | 4% | 28% |
| p30 | 5% | 35% |
| p31 | 17% | 38% |
| p34 | 16% | 41% |
| p39 | 29% | 66% |
| p41 | 34% | 53% |
| p45 | 5% | 35% |
| p58 | 5% | 35% |
| p66 | 7% | 39% |
| p93 | 32% | 60% |

Ticks are among the most important sources of vector-borne infections in the US. The spread of ticks across the US has been steadily increasing over the past decades. In parallel, the discovery of novel pathogens that are spread by ticks has also seen dramatic increases. Currently, there are 14 major tickborne diseases according to the CDC, namely, Lyme disease, babesiosis, ehrlichiosis, Rocky Mountain spotted fever, Southern tick-associated rash illness (STARI), hard tick relapsing fever, soft tick relapsing fever, tularemia, anaplasmosis, Colorado tick fever, bourbon virus, heartland virus, Rickettsia parkeri rickettsiosis, 364D rickettsiosis, and Powassan virus. Patients are rarely tested for all possible infections that could be transmitted via a tick bite. The current diagnostic tests are severely limited in distinguishing various tick-borne infections and several studies have revealed that non-Lyme disease tick-borne infections are heavily underdiagnosed

Tickborne infections can be challenging to diagnose since their symptoms can be nonspecific and overlap with other illnesses. The current diagnostic tests are severely limited in distinguishing various tick-borne infections. Several diagnostic tests are available to detect tick-borne infections. Of these, PCR and serology-based assays are reliable and widely used.

PCR has some advantages in that it detect pathogenic DNA/RNA which conclusively proves the organism's presence. It has high sensitivity and specificity and has a high throughput with assay run times of about 2 hours. Moreover, it can also detect the infection during its early stages. However, there are numerous drawbacks to testing using PCR since it requires special laboratories and equipment for testing. Pathogens transmitted by vectors may be transient in the blood resulting in clinically false negative PCR in tick-borne diseases and other infections, namely, B. burgdorferi, R. typhi, T. gondii, HSV-1, EBV, TBEV, and WNV. PCR testing requires specialized laboratories and equipment for testing. PCR may not detect all strains and variants and is limited to detecting known pathogens. Multiplexing with PCR is limited due to fixed number of analytes that can be parallelly read using PCR instrumentation.

Like PCR, serology-based testing has advantages and disadvantages. Serology-based testing has some advantages when it comes to tick-borne infection testing. Pathogens transmitted by ticks may be transient in the blood resulting in false negative PCR results in tick-borne diseases, namely B. burgdorferi, R. typhi, T. gondii, HSV-1, EBV, TBEV, and WNV. Serology-based testing has the ability to comprehensively assess immune responses and simultaneously detect multiple pathogens. It can also detect previous and unresolved infections. Moreover, serology-based assays are highly cost-effective, accessible, and can be used for epidemiological studies. However, serological studies have their own limitations. Serological testing may not be able to detect early or recent infections. It also relies heavily on the timing of sample collection and the host's immune responses. Molecular testing may be needed to confirm serological testing in certain cases. Despite all this, the benefits of serology testing outweigh its limitations which is why it is recommended by the CDC as the standard of testing for Lyme disease.

Serology-based multiplex testing for tickborne diseases involving the detection of specific antibodies produced by the immune system in response to tick-borne pathogens can be a valuable tool in diagnosing these infections. Simultaneous detection of antibodies against multiple tick-borne pathogens in a single sample and providing a comprehensive view of the patient's immune response is a key advantage of serology-based multiplex testing. The collection can also be in a dried blood spot. This can be particularly useful in areas with multiple tickborne diseases or when considering potential co-infections.

Serological testing can diagnose tick-borne diseases even in the later stages when pathogen detection through molecular methods becomes more challenging. It also reduces the risk of false negatives. Serological multiplex testing can also contribute to surveillance and epidemiological studies by providing valuable data on the prevalence and distribution of tick-borne diseases. The ability to simultaneously detect antibodies against multiple pathogens in a large number of samples can facilitate population-level studies and enhance understanding of disease dynamics.

Apart from PCR and ELISA serology tests, IFA and culture methods have also been suggested for diagnosing tick-borne infections. Testing using IFA is limited due to a lack of standardized antigenic targets, the subjective establishment of positive thresholds, and cross reactivity. These factors can result in varying accuracy of IFA results across laboratories. Furthermore, bacterial or viral cultures are not recommended for the diagnosis of tick-borne infections. This is due to the time-consuming nature of the test, the need for special media, and procedures that are only performed at specific laboratories.

This study employed a serology-based microarray developed to multiplex Lyme disease and other tick-borne and opportunistic infections of interest. The uniqueness of the microarray lies in the application of the immunodominant antigens that eliminate nonspecific binding with high sensitivity needed for accurate diagnosis (e.g., eliminates the nonspecific epitopes and false negatives). Antigens can be evaluated in a multiplex setting to gauge their performance with clinical samples to pick the ideal set of antigens for any infection. This is the first report on the broad panel of antigens for Lyme disease, co-infections, and other related infections in such a flexible format. The structure of the pillar plate is designed to encompass up to 400 probe chips at each pillar, facilitating the detection of an array of co-infections in a single experiment, resulting in cost, labor, and time savings. Further compaction of the chip allows improved performance by enhancing multiplexing and widening its clinical applications. The microarray platform is advantageous over other existing gold standards for tick-borne diseases and was able to overcome several of their limitations. Moreover, the average time for the two-tiered testing is approximately four days and for multitier testing for several tickborne pathogens can be up to several months, whereas the microarray technology disclosed herein takes about one day to perform. The microarray detected 17 tick-borne and other infections along with Lyme disease with sensitivities and specificities listed in Table 4.

A two-tiered testing algorithm (ELISA followed by western blot) is currently recommended for Lyme disease. Even though this method has high sensitivity and specificity in disseminated disease, its ability to detect the early stages of the disease is very limited. Around 28% of IgM western blots in two-tiered testing yielded false positives and could only detect less than 40% of individuals with early stages of the disease. Testing using IFA is limited due to a lack of standardized antigenic targets, the subjective establishment of positive thresholds, and cross reactivity. These factors can result in varying accuracy of IFA results across laboratories. In addition, bacterial or viral cultures are not recommended for the diagnosis of tick-borne infections due to the time-consuming nature of the test, the need for special media, and procedures that are only performed at specific laboratories.

The customizable protein microarray design includes probe molecules (e.g., antigens) physically separated by design, unlike blot assays, and can multiplex across species. Multiplexing can also be done across antigen types such as proteins, peptides, (e.g. recombinant protein and/or peptides) and lysates simultaneously. This method can lower test costs since all the manufacturing is automated using bio customized semiconductor processes similar to how electronic chips are made. The multiplex microarray has three main advantages over the existing technologies. First, it has an ultra-high-density array surface with high reproducibility and better throughput. Second, it can detect a large number of antibodies against varied infectious agents at the same time. And third, detection of antibodies can be performed using low sample volumes with low cost and a fast turnaround time. Given the flexible nature of the multiplex platform, provided herein is a multiplexed testing solution for Lyme disease, its co-infections, and other possible infections of interest.

In conclusion, the protein microarray with a multiplex of antigens disclosed herein has successfully diagnosed Lyme disease, co-infections, and other related infections in subject samples with high sensitivity and specificity. Simultaneous testing for Lyme disease, co-infections, and other related infections makes the diagnosis and treatment easier and quicker. This approach caters to the diagnostic needs of patients owing to its high sensitivity and specificity, affordable cost, quick availability of results, and low sample volume requirement. Measures for syndromic surveillance, diagnostic preparedness in disease outbreak investigations, personal protection, and education of clinical health professionals and patients could play a role in controlling tick-borne infections. As the known repertoire of antigens increases, this flexible microarray format can be customized to include the new antigens. Additionally, ticks have also been shown to spread Colorado tick fever, heartland virus, rickettsiosis, Rocky Mountain spotted fever, Southern tick-associated rash illness, tick-borne relapsing fever, tularemia, and Q fever. Microarrays can include other infections or pathogens, such as Colorado tick fever, heartland virus, Southern tick-associated rash illness, tick-borne relapsing fever, and tularemia which can be tested in parallel. Novel antigens for pathogens which may include whole cell lysates, recombinant proteins or peptide epitopes can be added as the science progresses leading to continuous improvement in diagnostic technology for detecting tick-borne infections. The protein microarray in its present form can multiplex up to 400 antigens simultaneously and its design is flexible, allowing for the addition of any new antigens.

### Example 2: Comparison with Single Pathogen Assays

Patient samples are tested with the microarray described in Example 1 and commercially available tests that assess only one pathogen. Commercially available tests used include, but are not limited to, the assays provided in Table 5. Table 5 includes the pathogen tested for, the vendor, a description of the test and the vendor catalogue number.

TABLE 5
Comparator Assays

| Pathogen | Vendor | Description | Catalog # |
|---|---|---|---|
| Borrelia | Euroimmun | EUROLINE Borrelia-RN-AT | DN 2131-3201 G |
| burgdorferi | | | |
| Babesia microti | Fuller | Babesia microti 12-well IFA Substrate | BM-12 |
| | Laboratories | Slides | |
| Babesia duncani | Fuller | Babesia duncani (WA-1) 12-well IFA | BD-12 |
| | Laboratories | Substrate Slides | |
| Anaplasma | Fuller | Anaplasma phagocytophilum IFA IgG Kit | EEG-120 |
| | Laboratories | | |
| Bartonella | Fuller | Bartonella henselae 12-well IFA Slides | BH-12 |
| henselae | Laboratories | | |
| Bartonella | Fuller | Bartonella quintana 12-well IFA Slides | BQ-12 |
| quintana | Laboratories | | |
| Ehrilichia | Fuller | Ehrlichia chaffeensis IFA IgG Kit | ECHG-120 |
| | Laboratories | | |
| Rickettsia | Fuller | Rickettsia rickettsii IFA IgG Test Kit | RRG-120 |
| rickettsii | Laboratories | | |
| Rickettsia typhi | Fuller | Rickettsia typhi IFA IgG Test Kit | RTG-120 |
| | Laboratories | | |
| TBEV | Euroimmun | Anti-TBE Virus ELISA 2.0 (IgG) | EI 2661-9601-2 G |
| West Nile virus | Inbios | West Nile Detect ™ IgM Capture ELISA | WNMS-1 |
| West Nile virus | Serion | West Nile Virus IgG | ESR141G |
| Chlamydophila | Serion | Chlamydia pneumoniae IgG and IgM | ESR1371G, |
| | | | ESR1371M |
| Coxsackie virus | Serion | Coxsackievirus IgG and IgM | ESR134G, |
| | | | ESR134M |
| Mycoplasma | Zeus | M. pneumoniae IgG and IgM Test System | 3Z17601G, |
| pneumoniae | Scientific | | 3Z17601M |
| Parvovirus | Zeus | Parvovirus B19 IgG Test System | 9Z7701G |
| | Scientific | | |
| Toxoplasma | Zeus | T. gondii IgG and IgM Test System | 8Z8651G, 8Z8651M |
| gondii | Scientific | | |
| HSV1 | Euroimmun | Anti-Herpes simplex virus 1 ChLIA (IgG) | LI 2531-10010 G |
| HSV2 | Zeus | HSV gG-2 IgG Test System | 9Z9781G |
| | Scientific | | |
| CMV | Zeus | CMV IgG and IgM Test System | 9Z9501G, 9Z9501M |
| | Scientific | | |
| EBV | Zeus | EBV-VCA IgG Test System | 9Z9201G |
| | Scientific | | |
| EBV | Zeus | EBV-VCA IgM Test System | 9Z9201M |
| | Scientific | | |
| EBV | Zeus | EBNA-1 IgG Test System | 9Z9461G |
| | Scientific | | |
| EBV | Zeus | EBV-EA IgG Test System | 9Z9481G |
| | Scientific | | |
| HHV6 | Fuller | HHV-6 12-well IFA Substrate Slides | H6-12 |
| | Laboratories | | |

The sensitivity and specificity of the microarray described herein and the commercial assays to diagnosis pathogen infections disclosed herein in Tables A, 1, 3, and 4 are compared. The microarray described herein is observed to have a higher sensitivity and specificity than the commercial assays, and thus better diagnostic ability than the commercial assays.

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

1. A microarray comprising a surface and two or more features attached to the surface, wherein the features comprise a probe molecule from one or more tickborne pathogens and wherein the probe molecules comprise two or more of a lysate, an antigen, a protein, or a peptide.

2. The microarray of claim 1, wherein the antigen comprises an antigenic fragment.

3. The microarray of claim 1 or 2, wherein the two or more features are distinct from each other.

4. The microarray of claims 1-3, wherein the two or more features comprise at least one lysate, at least one antigen, at least one protein, and/or at least one peptide.

5. The microarray of claim 4, wherein the antigen comprises an antigenic fragment.

6. The microarray of any one of claims 1-5, wherein the microarray is configured to have an at least 25%, 55%, 60%, 65%, 70%, 75%, 80%, 85%, 90%, 91%, 92%, 93%, 94%, 95%, 96%, 97%, 98%, 99%, or 100% sensitivity for detection of a tickborne pathogen after contact of the features with a sample from a subject suspected of having a tickborne pathogen.

7. The microarray any one of claims 1-6, wherein the microarray is configured to have an at least 90%, 91%, 92%, 93%, 94%, 95%, 96%, 97%, 98%, 99%, or 100% specificity for detection of a tickborne pathogen after contact of the features with a sample from a subject suspected of having a tickborne pathogen.

8. The microarray of claims 1-7, wherein the microarray is configured to have at least 90% sensitivity and 90% specificity for detection of a tickborne pathogen after contact of the features with a sample from a subject suspected of having a tickborne pathogen.

9. The microarray of claims 1-7, wherein the microarray is configured to have at least 93% sensitivity and 96% specificity for detection of a tickborne pathogen after contact of the features with a sample from a subject suspected of having a tickborne pathogen.

10. The microarray of claims 1-7, wherein the microarray is configured to have at least 95% sensitivity and 95% specificity for detection of a tickborne pathogen after contact of the features with a sample from a subject suspected of having a tickborne pathogen.

11. The microarray of any one of claims 1-10, wherein the one or more tickborne pathogens comprises Borrelia burgdorferi, Borrelia mayonii, Borrelia afzelii, Borrelia garinii, Borrelia Bavariensis, Borrelia speielmanii, Borrelia hemsii, Borrelia Turicatae, Borrelia miyamotoi, Borrelia andersonii, Borrelia maritima, Borrelia californiensis, Borrelia bissettiae, Borrelia lusitaniae, Borrelia valaisiana, Borrelia yangtzensis, Borrelia turcia, Borrelia lonestari, Babsia microti, Babsia duncani, Bartonella henselae, Anaplasma phagocytophilum, Ehrlichia chaffeensis, Rickettsia typhi, Rickettsia rickettsia, Rickettsia parkeri, Francisella tularensis, Powassan virus, Tick-borne encephalitis virus, West Nile virus, Coxsackie virus, Cytomegalovirus, Epstein Barr virus, Parvovirus B19, Toxoplasma, gondii, Colorado tick fever virus, Heartland virus, Bourbon virus, HSV-1, HSV-2, HHV-6, or HHV-7.

12. The microarray of claims 1-11, wherein the one or more tickborne pathogens is selected from Tables 1, 3, or A.

13. The microarray of any one of claims 1-12, wherein the one or more tickborne pathogen comprises Borrelia burgdorferi.

14. The microarray of any one of claims 1-13, wherein the microarray comprises two or more probe molecules from distinct tickborne pathogens.

15. The microarray of any one of claims 1-14, wherein the microarray comprises two or more features comprising probe molecules from at least 1, 2, 3, 4, 5, 6, 7, 8, 9, 10, 11, 12, 13, 14, 15, 16, 17, 18 or more tickborne pathogens.

16. The microarray of any one of claims 1-15, wherein the microarray comprises two or more features comprising probe molecules from the same tickborne pathogen.

17. The microarray of any one of claims 1-15, wherein the array comprises at least 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 32, 330, 340, 350, 360, 370, 380, 390, 400 or more features comprising probe molecules from one or more tickborne pathogen.

18. The microarray of any one of claims 1-17, wherein the microarray comprises at least four hundred or more features comprising probe molecules from one or more tickborne pathogen.

19. The microarray of any one of claims 1-18, wherein the two or more features comprising probe molecules comprise antigens or lysates selected from Table 3 or Table B.

20. The microarray of any one of claims 1-19, wherein the two or more features comprising probe molecules comprise:
a. B. burgdorferi VLsE1, C6 peptide, DbpB, OspC, p18, p32, p28, p30, p31, OspA, OspB, BmpA, p34, p39, P41, p45, p58, p66, p83, p84, p85 p86, p87, p88, p89, p90, p91, p92, p93, whole cell sonicate crude extract B31, or whole cell sonicate crude extract B297;
b. B. afzelii BmpA, DbpA, OspA, OscpC, or p100;
c. B. garinii DBpA or OspC;
d. B. bavariensis p58, VLsE1, DbpA;
e. B. spielmanii DBpA or OspC;
f. B. miyamotoi GlpQ;
g. Babersia microtia IRA, p32, p41,
h. Bartonella henselae 17 kDa, 26 kDa, or SucB;
i. Anaplasma phgocytophilum MSP5, MSP2, or OmpA;
j. Rickettsia typhi OmpB or surface antigen;
k. Cytomeglovirus EIA, gB, p150, p28, p52, pp65, or p38;
l. Epstein Barr virus EA, EBNA1, VCA, gp125, p18, or p23;
m. Parvovirus VLP VLP2, VLP VP1/VP2;
n. Toxoplasma gondii MIC3, p24, p29, or 30;
o. Rickettsia rickettsia rompA or rompB;
p. Rickettsia parkeri OmpA, OmpB, PS 120, or 17 kDa;
q. Francisella tularensis LPS O antigen;
r. Heartland virus Gn or Gc; and/or
s. a lysate derived from one or more of Borrelia burgdorferi, Borrelia mayonii, Borrelia afzelii, Borrelia garinii, Borrelia Bavariensis, Borrelia speielmanii, Borrelia hemsii, Borrelia Turicatae, Borrelia miyamotoi, Borrelia andersonii, Borrelia maritima, Borrelia californiensis, Borrelia bissettiae, Borrelia lusitaniae, Borrelia valaisiana, Borrelia yangtzensis, Borrelia turcia, Babsia microti, Bartonella henselae, Anaplasma phagocytophilum, Ehrlichia chaffeensis, Rickettsia typhi, Rickettsia rickettsia, Powassan virus, Tick-borne encephalitis virus, West Nile virus, Coxsackie virus, Cytomegalovirus, Epstein Barr virus, Parvovirus B19, Toxoplasma, gondii, HSV-1, HSV-2, HHV-6, HHV-7, Colorado tick fever virus, Bourbon virus, or Borrelia lonestari.

21. The microarray of any one of claims 1-19, wherein the two or more features comprising probe molecules comprise B. burgdorferi VLsE1, C6 peptide, B31, B297, p18, p28, p30, p31, p34, p39, P41, p45, p58, p66, and p93 antigens.

22. The microarray of any one of claims 1-20, wherein the one or more tickborne pathogens is carried by at least one tick species selected from Table A or Table 3.

23. The microarray of any one of claims 1-22, wherein the microarray comprises two or more features comprising probe molecules from one or more tick species selected from Table A or Table 3.

24. The microarray of any one of claims 1-23, wherein the micro array comprises two or more features comprising probe molecules from at least 1, 2, 3, 4, 5, 6, 7, 8, 9, 10, 11, 12, 13, 14, 15, 16, 17, 18, 19, 20, or more tick species.

25. The microarray of any one of claims 1-24, wherein the tick species is any one of Ixodes scpularis, Ixodes ricinus, Ixodes persulatus, Ixodes uriae, Ornithodoros hermsi, Ornithodoros turicatae, Ixodes dentatus, Ixodes pacificus, Ixodes spinipalpis, Ixodes jellisonii, Ixodes nippopensis, Ixodes columnae, Ixodes granulatus, Hyalomma aegypticum; Amblyomma Americanum, Dermacentor variabilis, Dermacentor andersoni, Haemaphysalis longicornis, Ixodes cookie, Ornithodoros moubata, Amblyomma americanum, Dermacentor variabilis, Dermacentor andersoni, Rhipicephalus sanguineus, Dermacentor similis, Amblyomma Americanum, Amblyomma maculatum, Hemaphysalis longicornis, or Dermacentor similis.

26. The microarray of any one of claims 1-25, wherein the tickborne pathogen generates an immune response in a subject.

27. The microarray of any one of claims 1-25, wherein the tickborne pathogen causes a disease in a subject.

28. The microarray of claim 26 or 27, wherein the tickborne disease is Lyme disease, rickettsiosis, Rocky Mountain spotted fever, Southern tick-associated rash illness, tick-borne relapsing fever, tularemia, or Q fever.

29. The microarray of any one of claims 26-28, wherein the subject is human.

30. The microarray of any one of claims 1-29, wherein the features are at positionally-defined locations on the surface.

31. The microarray of any one of claims 1-30, wherein the surface comprises one or more silicon wafer microchips.

32. The microarray of any one of claims 1-31, wherein the microarray comprises 10-100, 10-20, 20-30, 30-40, 40-50, 50-60, 60-70, 79-80, 80-90, 90-100, 100-150, 150-200, 200-250, 250-300, 300-350, 350-400, or more microchips, optionally 87 microchips.

33. The microarray of any one of claims 1-32, wherein the microarray is attached to a support surface.

34. The microarray of claim 33, wherein the microchips are at positionally-defined locations on the support surface.

35. The microarray of claim 33 or 34, wherein the support surface is a pillar plate.

36. The microarray of claim 35, wherein the pillar plate comprises a 12, 24, 36, 48, or 96 pillar plate.

37. A method for obtaining feature binding data, comprising:
a. obtaining the microarray of any one of claims 1-36,
b. contacting the microarray with a sample comprising a plurality of ligands for at least a subset of the features under conditions that promote ligand binding; and
c. imaging the microarray to identify binding of the plurality of ligands to the features of the microarray.

38. A method of identifying a tickborne disease or pathogen in a subject, comprising:
a. contacting a sample from the subject with the microarray of any one of claims 1-36; and
b. identifying binding of ligands in the sample to the features on the microarray to determine whether the subject has the tickborne disease or pathogen.

39. The method of claim 38, wherein the identifying binding of ligands in the sample comprises:
a. contacting the microarray with a sample comprising a plurality of ligands for at least a subset of the features under conditions that promote ligand binding; and
b. imaging the microarray to identify binding of the plurality of ligands to the features of the microarray.

40. The method of any one of claims 38-39, wherein the tickborne disease is Lyme disease, rickettsiosis, Rocky Mountain spotted fever, Southern tick-associated rash illness, tick-borne relapsing fever, tularemia, or Q fever.

41. The method of any one of claims 38-40, wherein the pathogen is Borrelia burgdorferi, Borrelia mayonii, Borrelia afzelii, Borrelia garinii, Borrelia Bavariensis, Borrelia speielmanii, Borrelia hemsii, Borrelia Turicatae, Borrelia miyamotoi, Borrelia andersonii, Borrelia maritima, Borrelia californiensis, Borrelia bissettiae, Borrelia lusitaniae, Borrelia valaisiana, Borrelia yangtzensis, Borrelia turcia, Borrelia lonestari, Babsia microti, Babsia duncani, Bartonella henselae, Anaplasma phagocytophilum, Ehrlichia chaffeensis, Rickettsia typhi, Rickettsia rickettsia, Rickettsia parkeri, Francisella tularensis, Powassan virus, Tick-borne encephalitis virus, West Nile virus, Coxsackie virus, Cytomegalovirus, Epstein Barr virus, Parvovirus B19, Toxoplasma, gondii, Colorado tick fever virus, Heartland virus, Bourbon virus, HSV-1, HSV-2, HHV-6, or HHV-7.

42. The method of any one of claims 37-41, wherein the sample is from a subject.

43. The method of any one of claims 37-42, wherein the sample is blood or serum.

44. The method of any one of claims 37-43 wherein the subject is human.

45. The method of any one of claims 37-44, wherein the ligands comprise an antibody or antigen-binding fragment.

46. The method of claim 45, wherein the ligand comprises an IgG antibody or IgM antibody or a combination thereof.

47. The method of any one of claims 37-46, wherein the method comprises identifying binding of IgG, IgM or IgG and IgM antibodies present in the sample to the probe molecules on the microarray.

48. The method of any one of claims 37-47, wherein the method comprises a sensitivity of detection of IgM antibodies that bind to the tickborne disease or pathogen with a sensitivity of at least 25%, 55%, 60%, 65%, 70%, 75%, 80%, 85%, 90%, 91%, 92%, 93%, 94%, 95%, 96%, 97%, 98%, or 99%.

49. The method of any one of claims 37-48, wherein the method comprises a specificity of detection of IgM antibodies that bind to the tickborne disease or pathogen with a sensitivity of at least 95%, 96%, 97%, 98%, 99%, or 100%.

50. The method of any one of claims 37-49, wherein the method comprises a sensitivity of detection of IgG antibodies that bind to the tickborne disease or pathogen with a sensitivity of at least 80%, 83%, 85%, 87%, 90%, 91%, 92%, 93%, 94%, 95%, 96%, 97%, 98%, 99%, or 100%.

51. The method of any one of claims 37-50, wherein the method comprises a specificity of detection of IgM antibodies that bind to the tickborne disease or pathogen with a sensitivity of at least 95%, 96%, 97%, 98%, 99%, or 100%.

52. The method of any one of claims 37-51, wherein the method comprises a sensitivity of detection of IgG and IgM antibodies that bind to the tickborne disease or pathogen with a sensitivity of at least 85%, 87%, 90%, 91%, 92%, 93%, 94%, 95%, 96%, 97%, 98%, 99%, or 100%.

53. The method of any one of claims 37-52, wherein the method comprises a specificity of detection of IgG and IgM antibodies that bind to the tickborne disease or pathogen with a sensitivity of at least 95%, 96%, 97%, 98%, 99%, or 100%.

54. The method of any one of claims 37-53, wherein a total number of features on the microarray is at least about 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 32, 330, 340, 350, 360, 370, 380, 390, or 400 or more.

55. The method of claims 37-54, wherein the microarray has an area that is less than or equal to 0.2 square millimeters (mm²), less than or equal to 0.7 square millimeters (mm²), less than or equal to 1 square millimeters (mm²), less than or equal to 10 square millimeters (mm²), less than or equal to 100 square millimeters (mm²), or less than or equal to 150 square millimeters (mm²).

56. The method of any one of claims 37-55, wherein the sample has a volume that is less than or equal to 100 μL, less than or equal to 50 μL, less than or equal to 25 μL, less than or equal to 10 μL, less than or equal to 5 μL, less than or equal to 1.5 μL, or less than or equal to 1 μL.

57. The method of any one of claims 37-56, wherein an elapsed time from sample contacting to finishing the imaging is equal or less than 20 minutes, equal or less than 5 minutes, equal or less than 1 minute, equal or less than 2-seconds, equal or less than 10 seconds, equal or less than 1 second.

58. The method of any one of claims 37-57, wherein a coefficient of variation of data obtained from the array is not greater than 5 percent, not greater than 2 percent, not greater than 1 percent.

59. The method of any one of claims 37-58, wherein the microarray comprises at least at least 1000 features per square centimeter, at least 5,000 features per square centimeter, at least 10,000 features per square centimeter, at least 50,000 features per square centimeter, at least 100,000 features per square centimeter, at least 500,000 features per square centimeter, at least 1,000,000 features per square centimeter, at least 10,000,000 features per square centimeter, or at least 15,000,000 features per square centimeter.

60. The method of any one of claims 37-59, the contacting occurs at a concentration of the plurality of ligands that is less or equal than 1,000 μg/ml in the sample, less or equal than 10 μg/ml in the sample, less or equal than 1 μg/ml in the sample, less or equal than 0.1 μg/ml in the sample, less or equal than 10 ng/ml in the sample, less or equal than 1 ng/ml in the sample, less or equal than 5 μg/ml in the sample.

61. The method of any one of claims 37-60, the contacting occurs at a concentration of the plurality of ligands that is within the range of approximately 1 μg/ml to approximately 1,000 μg/ml in the sample.

62. The method of any one of claims 37-61, wherein the imaging comprises identifying binding of at least 100 ligands, at least 500 ligands, at least 1,000 ligands, at least 100,000 ligands, at least 1,000,000 ligands, at least 10,000,000 ligands, at least 15,000,000 ligands, or at least 100,000,000 ligands to the features of the microarray.
